(19) United States
(12) Patent Application Publication
(10) Pub. No.: US 2022/0125372 A1
(43) Pub. Date: Apr. 28, 2022

(54) METHODS AND DEVICES FOR SCREENING SWALLOWING IMPAIRMENT

(71) Applicant: SOCIETE DES PRODUITS NESTLE S.A., Vevey (CH)

(72) Inventors: Michael Jedwab, Lausanne (CH); Simon Picthall, Borex (CH)

(21) Appl. No.: 17/429,055

(22) PCT Filed: Feb. 10, 2020
(86) PCT No.: PCT/EP2020/053251

§ 371 Date: Aug. 6, 2021

Related U.S. Application Data
(60) Provisional application No. 62/804,849, filed on Feb. 13, 2019.

(51) Int. Cl. A61B 5/00; A61B 7/00

(52) U.S. Cl. CPC A61B 5/4205; A61B 7/008; A61B 2562/0204; A61B 5/6822; A61B 5/742

(57) ABSTRACT

An integrated device for screening swallowing safety and swallowing efficiency can (i) receive first vibrational data for a first set of swallowing events executed successively by a first individual, (ii) compare at least a portion of the first vibrational data and/or at least a portion of second vibrational data derived from the first vibrational data against preset classification criteria defined for each of swallowing safety and swallowing efficiency, (iii) assign a swallowing safety probability and a swallowing efficiency probability to each of the first plurality of swallowing events, (iv) determine a swallowing safety classification based at least partially on the swallowing safety probability of each of the first plurality of swallowing events and (v) determine a swallowing efficiency classification based at least partially on the swallowing efficiency probability of each of the first plurality of swallowing events.

### BACKGROUND

The present disclosure generally relates to methods and devices that use vibrational data, such as acoustic data and/or accelerometry data, to screen for swallowing impairment. More specifically, an integrated device uses vibrational data for screening swallowing safety and swallowing efficiency.

Dysphagia is characterized by impaired involuntary motor control of swallowing process and can cause “penetration” which is the entry of foreign material into the airway. The airway invasion can be accompanied by “aspiration” in which the foreign material enters the lungs and can lead to serious health risks.

The three phases of swallowing activity are oral, pharyngeal and esophageal. The pharyngeal phase is typically compromised in patients with dysphagia. The impaired pharyngeal phase of swallowing in dysphagia is a prevalent health condition (38% of the population above 65 years) and may result in prandial aspiration (entry of food into the airway) and/or pharyngeal residues, which in turn can pose serious health risks such as aspiration pneumonia, malnutrition, dehydration, and even death. Swallowing aspiration can be silent (i.e., without any overt signs of swallowing difficulty such as cough), especially in children with dysphagia and patients with acute stroke, rendering detection via clinical perceptual judgement difficult.

Screening for dysphagia is a process used to identify those patients who are at risk for aspiration, malnutrition or dehydration and who need a further clinical assessment by a professional trained in the diagnosis and management of dysphagia. Screening alone is inadequate to detect the presence or absence of dysphagia or aspiration; however, patients with swallowing problems should be identified as early as possible to allow more severely impaired patients to be managed without delay. Screening for dysphagia is essential on initial admission to develop treatment plans, determine if food and fluids should be withheld from the patient, and whether a nasogastric (NG) tube will be necessary, as well as other issues related to eating and nutrition, aspirating, and swallowing food.

Although a wide variety of swallow screening and assessment tests are available for use, none have acceptable sensitivity and specificity to ensure accurate detection of dysphagia.

Several reviews have shown a lack of consensus regarding the best screening instrument to use. Most bedside swallowing examinations have been shown to lack sufficient sensitivity to be used for screening purposes, regardless of the patient populations examined. No bedside screening protocol has been shown to provide adequate predictive value for the presence of aspiration. Several individual exam components have demonstrated reasonable sensitivity, but reproducibility and consistency of these protocols was not established. Dysphagia screening validation studies reported in the literature have a number of serious limitations. It is also important to note that between one-third and one-half of patients who aspirate following stroke are silent aspirators (i.e., penetration of food below the level of the true vocal cords, without cough or any outward sign of difficulty).

In 2010, the Joint Commission (which accredits health care organizations and programs in the United States) withdrew the dysphagia screening performance standard for acute stroke because the National Quality Forum could not endorse the standard, stating that there are “no standards for what constitutes a valid dysphagia screening tool, and no clinical trials have been completed that identify the optimal swallow screening.” Dysphagia screening was removed from the “Get with the Guidelines” stroke guidelines. However, removal from the Joint Commission recommendations does not mean that screenings should not be performed; indeed the Joint Commission recommends further research to improve dysphagia screening methods.

Videofluoroscopic swallowing study (VFSS) has long been regarded as the clinical reference method (gold standard) in the assessment of dysphagia. VFSS dynamically visualizes the oral, pharyngeal and esophageal phases of swallowing. VFSS provides a comprehensive assessment of swallowing, determining not only whether the patient is aspirating but also enabling an analysis of the pathophysiological mechanisms leading to aspiration. Penetration, aspiration and residue are most commonly graded according to the rating scale of Rosenbek, et al. However, VFSS requires specialized equipment and staff and involves exposure to radiation. Some patients are poorly suited to VFSS, such as those who are medically fragile and may be unable to be transported to radiology (e.g., complex acute stroke patients and ICU patients).

Fiber-optic endoscopic evaluation of swallowing (FEES) is another instrumental assessment of swallowing, using a flexible nasolaryngoscope which is passed through the nares, over the velum into the pharynx. Recent studies suggest that FEES is a safe, reliable and predictive tool for dysphagia assessment patients with acute stroke. The main disadvantage of FEES compared to VFSS is that not the whole swallowing act is covered and furthermore the endoscopic view is impaired intradeglutitively for a short moment. FEES is now probably the most frequently used tool for objective dysphagia assessment in Germany. It allows evaluation of the efficacy and safety of swallowing, determination of appropriate feeding strategies, and assessment of the efficacy of different swallowing maneuvers. AHA/ASA-Endorsed Practice Guidelines Management of Adult Stroke Rehabilitation Care recommends considering fiber-optic endoscopic examination of swallowing (FEES) as an alternative to VFSS.

A clinical (or bedside) swallowing evaluation (CSE) is a behavioral assessment of swallowing function usually performed by an SLP (Speech Language Pathologist). This evaluation is a practical method of assessment but has limitations and relies on subjective evaluation by skilled clinicians. 40% of variables typically used in a CSE are unsupported by data, and only 44% of the measures typically used by clinicians have exhibited adequate intra- and inter-judge reliability.

Bedside screening tests for dysphagia are safe, relatively straightforward, and easily repeated but have variable sensitivity (42% to 92%), specificity (59% to 91%), and interrater reliability (κ 0 to 1.0). They are also poor at detecting silent aspiration. The accuracy of the WST (Water Swallow Test), which is currently the tool used most often to screen patients at risk of dysphagia in clinical settings, has been repeatedly questioned during recent years. Two meta-analyses conducted by Ramsey, et al. and Bours, et al. suggested that when compared to VFSS or FEES, the sensitivity of the WST for detecting aspiration is markedly below 80% in nearly all reviewed studies. This observation also applies to specificity and also negative and positive predictive values.

Most swallow screening approaches involve observations of voice quality, voluntary cough function, speech clarity, tongue function and swallows of water or other stimuli. The clinicians administering the test are expected to identify abnormalities in these parameters, including post-swallow cough or wet voice. Blinded comparison of results between a standardized swallowing screening protocol in which clinicians were asked to judge these parameters and simultaneous VFSS revealed that none of the screening parameters were adequate for decision-making, for detecting pharyngeal dysphagia, or for detecting laryngeal penetration and aspiration.

The Toronto Bedside Swallowing Screening Test (TOR-BSST) reports a sensitivity of 91% (95% CI, 71.9-98.7) and a specificity of 67% (95% CI, 49.0-81.4). The limitations of this test include questionable feasibility, limited operational definitions, a small validation sample with only 20% of subjects (n=68) in the trial contributing to validation, and extended time between the screening and reference test. This last limitation is especially important in the stroke population due to the rapid evolution of dysphagia, especially in the acute period. The Gugging Swallow Screen (GUSS) was validated in a small study in acute stroke patients (delivered by SLPs in 19 patients and by nurses in 30 patients) and showed excellent sensitivity of 100% and specificity 50% for SLP validation and sensitivity of 100% and specificity of 69% in validation with nurses. The limitations include a lack of reliability information for nurses, unknown feasibility given complexity (the test consists of two parts including 3 sequentially performed subtests, starting with semisolid food, then liquids, and finally solid textures), and a small sample size in the validation study.

Notably, most screening methods have been developed for and tested in stroke patients. The clinical usability and accuracy of these methods in other populations at risk of dysphagia can be questioned. For example, the results of a self-administered survey from 836 certified SLPs from all fifty states in the U.S.A. showed that even though respondents reported being regularly involved in swallowing assessment and the provision of care for those who have received mechanical ventilation, the majority of SLP diagnostic evaluations (60%; 95% CI=59-62%) were performed using clinical techniques with uncertain accuracy.

Considering the limitations of the clinical swallowing examination, the CSE cannot be used as a reference method for the validation of new screening tools, leaving VFSS and FEES as only the only valid reference standards of choice.

The development of a fully automated, accurate swallowing screening tool remains an elusive challenge.

### SUMMARY

In a general embodiment, the present disclosure provides an integrated device for screening swallowing safety and swallowing efficiency. The device comprises a processor configured to:

(i) receive first vibrational data (e.g., acoustic data and/or accelerometry data) for a first plurality of swallowing events executed successively by a first individual (e.g., a single test such as a water test with four sips or a honey test with three sips),

(ii) compare swallowing data selected from the group consisting of at least a portion of the first vibrational data, at least a portion of second vibrational data derived from the first vibrational data, and a combination thereof against preset classification criteria defined for each of swallowing safety and swallowing efficiency,

(iii) assign a swallowing safety probability and a swallowing efficiency probability to each of the first plurality of swallowing events, each of the first plurality of swallowing events is assigned the corresponding swallowing safety probability and the corresponding swallowing efficiency probability independently from the other swallowing events to provide independent point measurements for the first plurality of swallowing events,

(iv) determine a swallowing safety classification based at least partially on the swallowing safety probability of each of the first plurality of swallowing events, the swallowing safety classification is identified from at least one predetermined swallowing safety classification, and preferably the swallowing safety classification is the single swallowing safety classification for the first plurality of swallowing events, and

(v) determine a swallowing efficiency classification based at least partially on the swallowing efficiency probability of each of the first plurality of swallowing events, the swallowing efficiency classification is identified from at least one predetermined swallowing efficiency classification, and preferably the swallowing efficiency classification is the single swallowing efficiency classification for the first plurality of swallowing events.

The device further comprises a user interface configured to provide one or more first outputs comprising at least one of audio and/or graphics that identify the swallowing safety classification and the swallowing efficiency classification for the first plurality of swallowing events.

An advantage of one or more embodiments provided by the present disclosure is an objective and non-invasive method to detect impaired swallowing in a patient at risk of oropharyngeal dysphagia of non-congenital and non-surgical and non-oncological origin.

Another advantage of one or more embodiments provided by the present disclosure is a screening test having a high sensitivity and high negative predictive value.

Yet another advantage of one or more embodiments provided by the present disclosure is a screening method for which the operating characteristics are established against a clinical reference standard as a validated, accurate and reliable dysphagia assessment method.

Still another advantage of one or more embodiments provided by the present disclosure is a swallowing screening device having the highest possible sensitivity with an acceptable specificity level.

Another advantage of one or more embodiments provided by the present disclosure is a screening test to detect silent aspiration.

### BRIEF DESCRIPTION OF THE FIGURES

FIG. 1 is diagram showing the axes of acceleration in the anterior-posterior and superior-inferior directions.

FIG. 2 is a schematic diagram of an embodiment of a device for screening swallowing impairment during operation.

FIG. 3A is an example of an interface screen for selecting between water and thickened beverage in a first embodiment of a device for screening swallowing impairment.

FIG. 3B is an example of an interface screen for entering patient information and displaying instructions in the first embodiment of a device for screening swallowing impairment.

FIG. 3C is an example of an interface screen for accepting user input, i.e., user input that identifies administration of a first bolus, in the first embodiment of a device for screening swallowing impairment.

FIG. 3D is an example of an interface screen for accepting user input, i.e., user input that identifies completion of the swallow of the first bolus, in the first embodiment of a device for screening swallowing impairment.

FIG. 3E is an example of an interface screen showing a swallowing safety classification and a swallowing efficiency classification of the first bolus and for accepting user input, i.e., user input that identifies administration of a second bolus, in the first embodiment of a device for screening swallowing impairment.

FIG. 3F is an example of an interface screen for accepting user input, i.e., user input that identifies completion of the swallow of the second bolus, in the first embodiment of a device for screening swallowing impairment.

FIG. 3G is an example of an interface screen showing a swallowing safety classification and a swallowing efficiency classification of the second bolus in the first embodiment of a device for screening swallowing impairment.

FIG. 3H is an example of an interface screen showing the swallowing safety classifications and the swallowing efficiency classifications of the first and second boluses and for accepting user input, i.e., user input that identifies administration of a third bolus, in the first embodiment of a device for screening swallowing impairment.

FIG. 3I is an example of an interface screen for accepting user input, i.e., user input that identifies completion of the swallow of the third bolus, in the first embodiment of a device for screening swallowing impairment.

FIG. 3J is an example of an interface screen showing the swallowing safety classifications and the swallowing efficiency classifications of the first, second and third boluses and for accepting user input, i.e., user input that identifies administration of a fourth bolus, in the first embodiment of a device for screening swallowing impairment.

FIG. 3K is an example of an interface screen for accepting user input, i.e., user input that identifies completion of the swallow of the fourth bolus, in the first embodiment of a device for screening swallowing impairment.

FIG. 3L is an example of an interface screen showing the swallowing safety classifications and the swallowing efficiency classifications of the first, second, third and fourth boluses in the first embodiment of a device for screening swallowing impairment.

FIG. 3M is an example of a summary screen in the first embodiment of a device for screening swallowing impairment.

FIG. 3N is an example of an interface screen for selecting a specific type of thickened beverage in the first embodiment of a device for screening swallowing impairment.

FIG. 3O is an example of an interface screen for entering patient information and displaying instructions in the first embodiment of a device for screening swallowing impairment.

FIG. 3P is an example of an interface screen for accepting user input, i.e., user input that identifies administration of a first bolus, in the first embodiment of a device for screening swallowing impairment.

FIG. 3Q is an example of an interface screen for entering patient information and displaying instructions in the first embodiment of a device for screening swallowing impairment.

FIG. 3R is an example of an interface screen for accepting user input, i.e., user input that identifies administration of a first bolus, in the first embodiment of a device for screening swallowing impairment.

FIG. 4A is an example of an interface screen for selecting between water and thickened beverage in a second embodiment of a device for screening swallowing impairment.

FIG. 4B is an example of an interface screen for entering patient information and displaying instructions in the second embodiment of a device for screening swallowing impairment.

FIG. 4C is an example of an interface screen for accepting user input, i.e., user input that identifies administration of a first bolus, in the first embodiment of a device for screening swallowing impairment.

FIG. 4D is an example of an interface screen for accepting user input, i.e., user input that identifies completion of the swallow of the first bolus, in the second embodiment of a device for screening swallowing impairment.

FIG. 4E is an example of an interface screen for accepting user input, i.e., user input that identifies administration of a second bolus, in the second embodiment of a device for screening swallowing impairment.

FIG. 4F is an example of an interface screen for accepting user input, i.e., user input that identifies completion of the swallow of the second bolus, in the second embodiment of a device for screening swallowing impairment.

FIG. 4G is an example of an interface screen for accepting user input, i.e., user input that identifies administration of a third bolus, in the second embodiment of a device for screening swallowing impairment.

FIG. 4H is an example of an interface screen for accepting user input, i.e., user input that identifies completion of the swallow of the third bolus, in the second embodiment of a device for screening swallowing impairment.

FIG. 4I is an example of an interface screen showing an analysis error for the third bolus in the second embodiment of a device for screening swallowing impairment.

FIG. 4J is an example of an interface screen for accepting user input, i.e., user input that identifies administration of a fourth bolus, in the second embodiment of a device for screening swallowing impairment.

FIG. 4K is an example of an interface screen for accepting user input, i.e., user input that identifies completion of the swallow of the fourth bolus, in the second embodiment of a device for screening swallowing impairment.

FIG. 4L is an example of an interface screen showing completion of swallowing of the first, second, third and fourth boluses in the second embodiment of a device for screening swallowing impairment.

FIG. 4M is an example of an interface screen showing a single swallowing safety classification and a single swallowing efficiency classification in the second embodiment of a device for screening swallowing impairment.

FIG. 4N is an example of a summary screen in the second embodiment of a device for screening swallowing impairment.

FIG. 4O is an example of an interface screen for selecting a specific type of thickened beverage in the second embodiment of a device for screening swallowing impairment.

FIG. 5 is a schematic diagram of an embodiment of a method of screening swallowing impairment.

FIGS. 6A and 6B are schematic diagrams of a methodology for identifying the first four matched pairs (i.e., the first four swallowing events measurable by both VFSS and accelerometry) in an embodiment of a method of screening swallowing impairment.

FIG. 7 is a photograph of an embodiment of a device for screening swallowing impairment.

FIG. 8 is a schematic diagram of basic architecture for measurement signal processing and transmission in an embodiment of a device for screening swallowing impairment.

FIG. 9 is a flowchart depicting participant progress through the clinical trial disclosed herein.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used in this disclosure and the appended claims, the singular forms “a,” “an” and “the” include plural referents unless the context clearly dictates otherwise. An example of context that dictates otherwise is the term “single” which herein means “only one,” e.g., a “single swallowing safety classification” and a “single swallowing efficiency classification” for a set of data respectively mean “only one swallowing safety classification” and “only one swallowing efficiency classification” for that set of data and exclude the presence of additional swallowing safety classifications and additional swallowing efficiency classifications for that set of data.

As used herein, “about” is understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, all numerical ranges herein should be understood to include all integers, whole or fractions, within the range.

The words “comprise,” “comprises” and “comprising” are to be interpreted inclusively rather than exclusively. Likewise, the terms “include,” “including” and “or” should all be construed to be inclusive, unless such a construction is clearly prohibited from the context. A disclosure of a device “comprising” several components does not require that the components are physically attached to each other in all embodiments.

Nevertheless, the devices disclosed herein may lack any element that is not specifically disclosed. Thus, a disclosure of an embodiment using the term “comprising” is also a disclosure of embodiments “consisting essentially of” and “consisting of” the components identified. Similarly, the methods disclosed herein may lack any step that is not specifically disclosed herein. Thus, a disclosure of an embodiment using the term “comprising” is also a disclosure of embodiments “consisting essentially of” and “consisting of” the steps identified.

The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive. Any embodiment disclosed herein can be combined with any other embodiment disclosed herein unless explicitly stated otherwise.

Numerical adjectives, such as “first” and “second,” are merely used to distinguish components. These numerical adjectives do not imply the presence of other components, a relative positioning, or any chronological implementation. In this regard, the presence of “second accelerometry data” does not imply that “first accelerometry data” is necessarily present. Further in this regard, “second accelerometry data” can be obtained and/or used before, after, or simultaneously with any “first accelerometry data.”

The terms “after,” “then” and “subsequent” merely mean that the event occurs at a time following the referenced event. These terms do not imply that the event occurs immediately following the referenced event, and any amount of time can elapse for an event to still be “after” the referenced event. Furthermore, these terms do not imply the absence of an intervening event, although this situation (e.g., “directly after” or “immediately after”) is encompassed by the terms “after,” “then” and “subsequent.”

As used herein, a “bolus” is a single sip or mouthful or a food or beverage. As used herein, “aspiration” is entry of food or drink into the trachea (windpipe) and lungs and can occur during swallowing and/or after swallowing (post-deglutitive aspiration). Post-deglutitive aspiration generally occurs as a result of pharyngeal residue that remains in the pharynx after swallowing.

As used herein, “swallowing safety” means the amount of a dose of beverage in a swallowing event that reaches the stomach relative to the amount of the dose of beverage that reaches the lungs, if any. “Swallowing efficiency” means how much beverage residue is left behind in the throat and/or by the lungs if any, after a swallowing event relative to the total dose of the beverage. “Swallowing inefficiency” is defined as the presence of visible residue in the pharynx at the end of any swallow, filling at least 50% of either the valleculae and/or the pyriform sinuses.

As used herein, “real-time” means the output is provided within ten seconds of the in a swallowing event, preferably within five seconds, more preferably within two seconds, most preferably within one second.

### Embodiments

An aspect of the present disclosure is an integrated device for screening swallowing safety and swallowing efficiency. Another aspect of the present disclosure is a method of screening swallowing safety and swallowing efficiency.

In some embodiments, the method and the device can be employed in one or more of the apparatus and/or the method for detecting aspiration disclosed in U.S. Pat. No. 7,749,177 to Chau et al., the method and/or the system of segmentation and time duration analysis of dual-axis swallowing accelerometry signals disclosed in U.S. Pat. No. 8,267,875 to Chau et al., the system and/or the method for detecting swallowing activity disclosed in U.S. Pat. No. 9,138,171 to Chau et al., or the method and/or the device for swallowing impairment detection disclosed in U.S. Pat. No. 9,687,191 to Chau et al., each of which is incorporated herein by reference in its entirety.

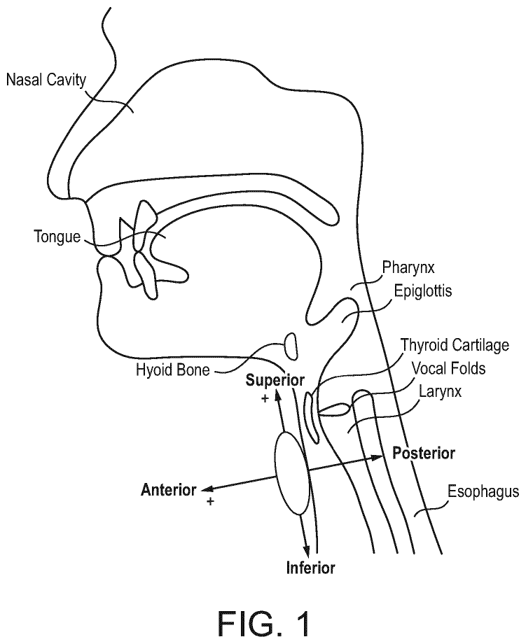

As discussed in greater detail hereafter, the device may include a sensor configured to produce signals indicating swallowing activities (e.g., a dual axis accelerometer or an acoustic sensor). The sensor may be positioned externally on the neck of a human, preferably anterior to the cricoid cartilage of the neck. A variety of means may be applied to position the sensor and to hold the sensor in such position, for example double-sided tape. Preferably the positioning of the sensor is such that the axes of acceleration are aligned to the anterior-posterior and super-inferior directions, as shown in FIG. 1.

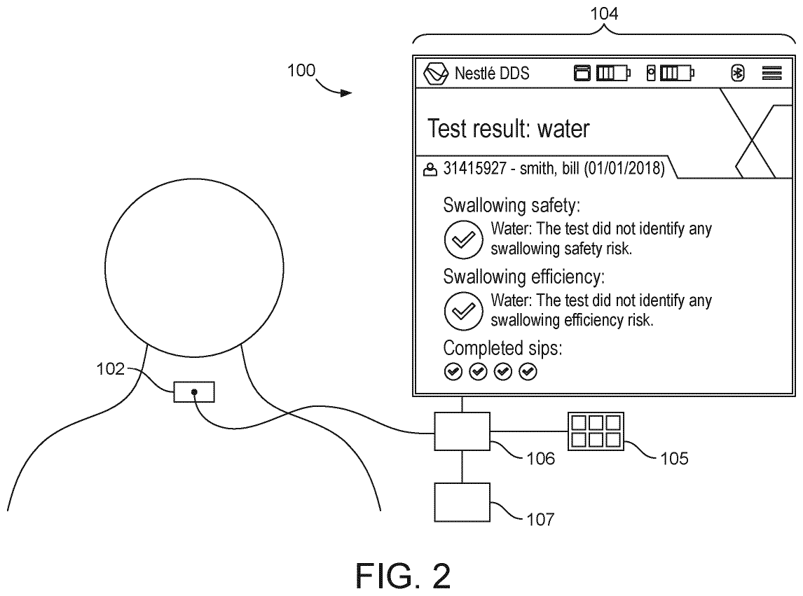

FIG. 2 generally illustrates a non-limiting example of a device 100 for screening swallowing safety and swallowing efficiency. The device 100 can comprise a sensor 102 (e.g., a dual axis accelerometer or an acoustic sensor) to be attached in a throat area of a candidate for acquiring vibrational data during swallowing, e.g., dual axis accelerometry data and/or signals. Accelerometry data may include, but is not limited to, throat vibration signals acquired along the anterior-posterior axis (A-P) and/or the superior-inferior axis (S-I). The sensor 102 can be any accelerometer known to one of skill in this art, for example a single axis accelerometer (which can be rotated on the patient to obtain dual-axis vibrational data) such as an EMT 25-C single axis accelerometer or a dual axis accelerometer such as an ADXL322 or ADXL327 dual axis accelerometer. The present disclosure is not limited to a specific embodiment of the sensor 102. Further in this regard, one of skill will understand that the disclosures herein regarding accelerometry data are also applicable to other vibrational data such as acoustic data and can be performed using other vibrational data such as acoustic data.

The sensor 102 can be operatively coupled to one or more processors 106 (hereafter “the processor 106” although any number of processors is contemplated). The processor 106 is configured to process the acquired data to determine swallowing efficiency and swallowing safety. The processor 106 can be a distinctly implemented device operatively coupled to the sensor 102 for communication of data thereto, for example, by one or more data communication media such as wires, cables, optical fibers, and the like and/or by one or more wireless data transfer protocols. In some embodiments, the processor 106 may be implemented integrally with the sensor 102.

Generally, the processing of the vibrational data (e.g., dual-axis accelerometry signals) comprises at least one of (i) a process in which at least a portion of the A-P signal and at least a portion of the S-I signal are analyzed individually by calculating the meta-features of each signal separately from the other channel or (ii) a process combining at least a portion of the axis-specific vibrational data for the A-P axis with at least a portion of the axis-specific vibrational data for the S-I axis and then extracting meta-features from the combined data.

The processor 106 of the device 100 is preferably configured to receive first vibrational data (e.g., accelerometry data or acoustic data) for a first plurality of swallowing events executed successively by a first individual. The sensor 102 of the device 100 can be an accelerometer communicatively connected to the processor 102 to provide the first vibrational data for the first plurality of swallowing events.

In an embodiment, the processor 106 uses one or more timing thresholds. For example, a first timing threshold can be applied when the device 100 is ready for receipt of the first vibrational data. In this regard, the device 100 can indicate readiness for receipt of the first vibrational data from one of the first plurality of swallowing events, and then the processor 106 can use the first timing threshold for receipt of user input indicating that the corresponding bolus is administered (e.g., selection of a “start” button). As non-limiting examples, the device 100 can indicate readiness for receipt of the first vibrational data and then provide fifteen minutes, preferably ten minutes, more preferably five minutes, for the user to enter input into the device 100 indicating that the corresponding bolus is administered. In a particular embodiment, the device 100 can provide an error message and/or stop the screening if the first timing threshold is exceeded before receiving the user input indicating that the corresponding bolus is administered.

Additionally or alternatively, a second timing threshold can be applied after the user input indicating that the corresponding bolus is administered. In this regard, the device 100 can accept the user input indicating that the corresponding bolus is administered (e.g., selection of a “start” button), and then the processor 106 can use the second timing threshold for receipt of user input indicating that the corresponding swallowing event is completed. As non-limiting examples, the device 100 can receive the user input indicating that the corresponding bolus is administered and then provide five minutes, preferably one minute, more preferably thirty seconds (or even less) for the user to enter input into the device 100 indicating that the corresponding swallowing event is completed. In a particular embodiment, the device 100 can provide an error message and/or stop the screening if the second timing threshold time is exceeded before the user input indicating that the corresponding swallowing event is completed.

The device 100 can further comprise a housing. The processor 106 can be positioned within the housing and/or mechanically connected to the housing. The device 100 preferably further comprises a user interface 104 which can be positioned within the housing and/or mechanically connected to the housing, and the user interface 104 can comprise an input element 105 (e.g., a keyboard or touchpad). The input element 105 can be configured to accept user input identifying screening parameters, such as a type of sensor that provides the accelerometry data and/or a type of beverage administered to the patient during the screening. The device 100 preferably further comprises a memory element 107 which can be positioned within the housing and/or mechanically connected to the housing.

Bolus-Level Analysis

In a first embodiment of the swallowing screening that can be performed by the device 100, the processor 106 can classify each swallowing event of the first plurality of swallowing events that are executed successively. Classification of each swallowing event is “bolus-level” analysis herein.

In this embodiment, each swallowing event can be classified based on the extracted meta-features of the corresponding swallowing event. In applying this approach, the swallowing events may be effectively classified as a normal swallowing event or a potentially impaired swallowing event (e.g., unsafe and/or inefficient). Preferably the classification is automatic such that no user input is needed for the dual-axis accelerometry signals to be processed and used for classification of the swallow event by the device 100. Each of the swallowing events can be classified independently from the other swallowing events to provide independent point measurements for the first plurality of swallowing events, therefore can be done in any order, and therefore can be used to monitor. Preferably, classification by the processor 106 of the plurality of swallowing events is real-time relative to the corresponding swallowing event.

In the first embodiment, the processor 106 can compare swallowing data (e.g., at least a portion of the first accelerometry data and/or at least a portion of second accelerometry data derived from the first accelerometry data) against preset classification criteria defined for each of swallowing safety and swallowing efficiency. The processor 106 can classify each of the first plurality of swallowing events with a swallowing safety classification and a swallowing efficiency classification based at least partially on the comparing of the swallowing data against the preset classification criteria. The swallowing safety classification is identified from at least one predetermined swallowing safety classification, and the swallowing efficiency classification is identified from at least one predetermined swallowing efficiency classification. The processor 106 can output the classifications, preferably on the user interface 104 of the device 100.

In the first embodiment, each of the first plurality of swallowing events can be classified independently from the other swallowing events to provide independent point measurements for the first plurality of swallowing events. Preferably, classification by the processor 106 of each of the first plurality of swallowing events is real-time relative to the corresponding swallowing event.

In the first embodiment, the user interface 104 of the device 100 is preferably configured to provide one or more first outputs comprising at least one of audio and/or graphics that identify the swallowing safety classification and the swallowing efficiency classification for each of the first plurality of swallowing events. Preferably, the one or more first outputs by the user interface 104 are each real-time relative to the corresponding swallowing event.

In the first embodiment, the processor 106 can be configured to use the user interface 104 to identify the swallowing safety classification and the swallowing efficiency classification for the first swallowing event simultaneously relative to each other. In an embodiment, the processor 106 is configured to use the user interface 104 to provide one or more second user outputs comprising at least one of audio and/or graphics that instruct administration of a plurality of doses of beverage, and each of the first plurality of swallowing events correspond to one of the plurality of doses of beverage.

For example, the processor 106 can be configured to use the user interface 104 to instruct administration of a first dose of beverage (and optionally subsequently accept user input indicating that swallowing of the first dose is completed). Then the user interface 104 can identify the swallowing safety classification and the swallowing efficiency classification for a first swallowing event corresponding to the first dose of beverage. Then the user interface 104 can instruct administration of a second dose of beverage (and optionally subsequently accept user input indicating that swallowing of the second dose is completed). Then the user interface 104 can identify the swallowing safety classification and the swallowing efficiency classification for a second swallowing event corresponding to the second dose of beverage.

In the first embodiment, the processor 106 can be configured to use the user interface 104, after identifying the swallowing safety classification and the swallowing efficiency classification for the second swallowing event, to instruct administration of a third dose of beverage (and optionally subsequently accept user input indicating that swallowing of the third dose is completed). Then the user interface 104 can identify the swallowing safety classification and the swallowing efficiency classification for a third swallowing event corresponding to the third dose of beverage. The processor 106 can be configured to use the user interface 104, after identifying the swallowing safety classification and the swallowing efficiency classification for the third swallowing event, to instruct administration of a fourth dose of beverage (and optionally subsequently accept user input indicating that swallowing of the fourth dose is completed). Then the user interface 104 can identify the swallowing safety classification and the swallowing efficiency classification for a fourth swallowing event corresponding to the fourth dose of beverage. In some embodiments, this bolus-level analysis can be performed for up to six boluses or even more.

In the first embodiment, the at least one predetermined swallowing safety classification can comprise a first swallowing safety classification indicative of a safe event and a second swallowing safety classification indicative of an unsafe event. The at least one predetermined swallowing efficiency classification can comprise a first swallowing efficiency classification indicative of an efficient event and a second swallowing efficiency classification indicative of an inefficient event. The one or more first outputs can comprise at least one icon displayed on the user interface 104 for each of the first plurality of swallowing events. At least a portion of the at least one icon can be a first color for the first swallowing safety classification (e.g., green) or a second color different than the first color for the second swallowing safety classification (e.g., red). At least a portion of the at least one icon can be a third color for the first swallowing efficiency classification (e.g., green) or a fourth color different than the third color for the second swallowing efficiency classification (e.g., red).

In the first embodiment, the memory element 107 can store and/or can upload the swallowing safety classification and the swallowing efficiency classification for each of the first plurality of swallowing events in a first profile associated with the first individual. The device 100 can be used to monitor the first individual by periodically screening the first individual and saving the results of the periodic screenings in the memory element 107.

For example, the processor 106 can be configured to screen a second plurality of swallowing events executed by the first individual subsequent to the first plurality of swallowing events, for example at least one day later, at least one week later, at least one month later, or at least one year later. The processor 106 can be configured to compare the swallowing safety and efficiency classifications for the first plurality of swallowing events to the swallowing safety and efficiency classifications for the second plurality of swallowing events. In some embodiments, such periodic comparisons can be used to monitor the relative progress or decline of a patient.

In the first embodiment, the device 100 can screen the individual for swallowing safety and swallowing efficiency for each of a plurality of beverages, such as one or more of water (50 mPa·s or less, e.g. 1 mPa·s), nectar (51-350 MPa·s), honey (351-1750 mPa·s) or pudding (>1750 mPa·s), and most preferably screen each type of beverage separately (i.e., initially screen one or more boluses of a first beverage, then screen one or more boluses of a second beverage). The plurality of beverages can be screened in any order. The device 100 can screen the first individual for one or more types of beverages at a first time and then screen the individual for the one or more types of beverages again periodically thereafter, for example at least one day, at least one week, at least one month, or at least one year between screenings.

For example, the first plurality of swallowing events can be executed on a first beverage having a first viscosity. The processor 106 can be configured to screen a second plurality of swallowing events executed by the first individual subsequent to the first plurality of swallowing events, and the second plurality of swallowing events can be executed on a second beverage having a second viscosity different than the first viscosity. The processor 106 can be configured to store (e.g., in the memory element 107) the swallowing safety classification and the swallowing efficiency classification for each of the first plurality of swallowing events in association with identification of the first beverage in the first profile associated with the first individual. The processor 106 can be configured to store (e.g., in the memory element 107) the swallowing safety classification and the swallowing efficiency classification for each of the second plurality of swallowing events in association with identification of the second beverage in the first profile associated with the first individual.

The device 100 can be used to screen and/or monitor a plurality of individuals, e.g., the first individual, a second individual, and optionally additional individuals. Preferably the individuals are screened autonomously (i.e., the screening results are separate for each individual relative to the screening results of the other individuals). Each of the plurality of individuals can have their own profile and preferably can be screened the same day as the other individuals if desired. The device 100 can be linked to a cloud and thus use the data from the plurality of individuals to build meta-analysis and improve the algorithm.

For example, the processor 106 can be configured to screen a second plurality of swallowing events executed by a second individual different than the first individual subsequent to the first plurality of swallowing events. The device 100 can store (e.g., in the memory element 107) the swallowing safety classification and the swallowing efficiency classification for each of the second plurality of swallowing events in a second profile associated with the second individual. The processor 106 can be configured to compare the swallowing safety and efficiency classifications for the first individual to the swallowing safety and efficiency classifications for the second individual, preferably while recording similarities or differences in characteristics of the individual, such as one or more of age, gender, height, weight and medical condition.

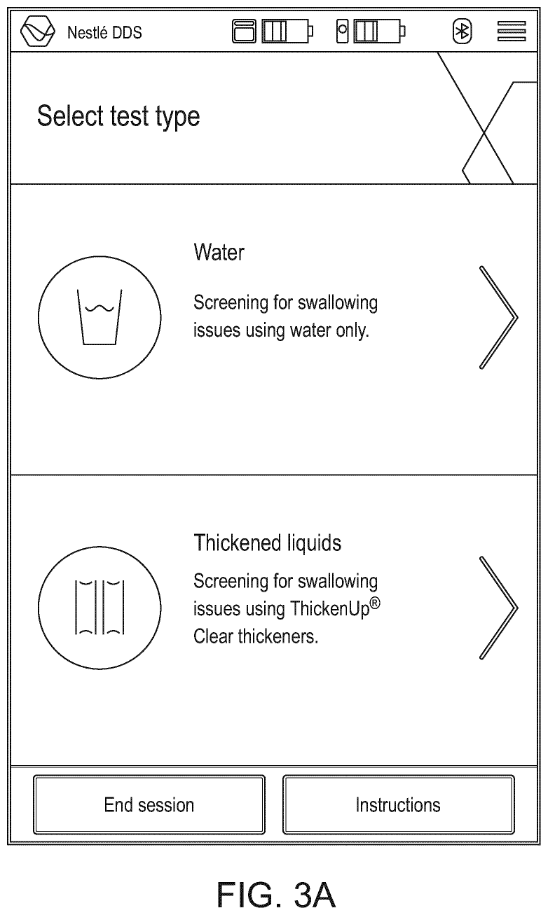

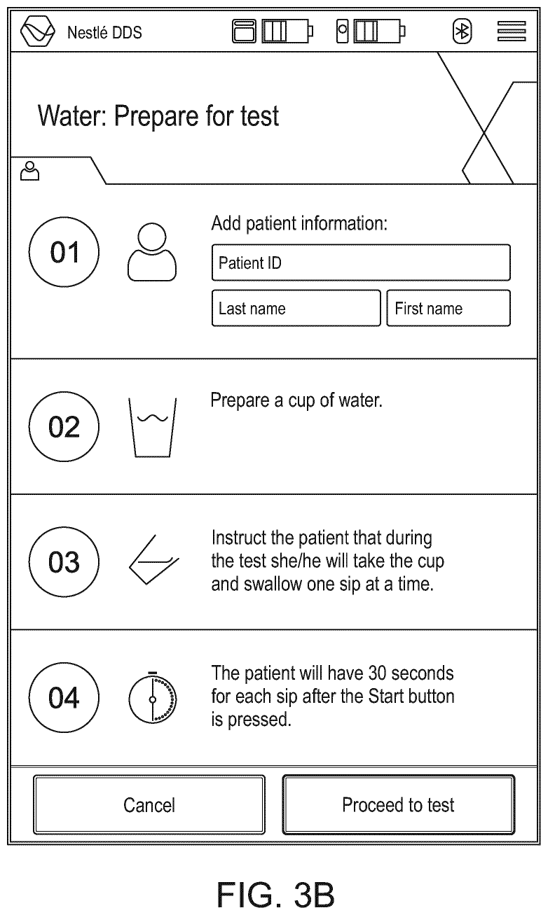

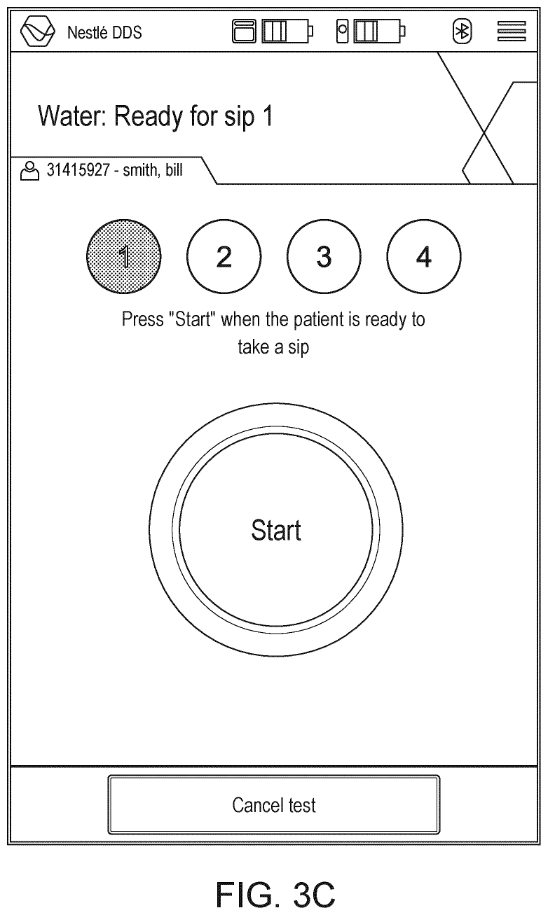

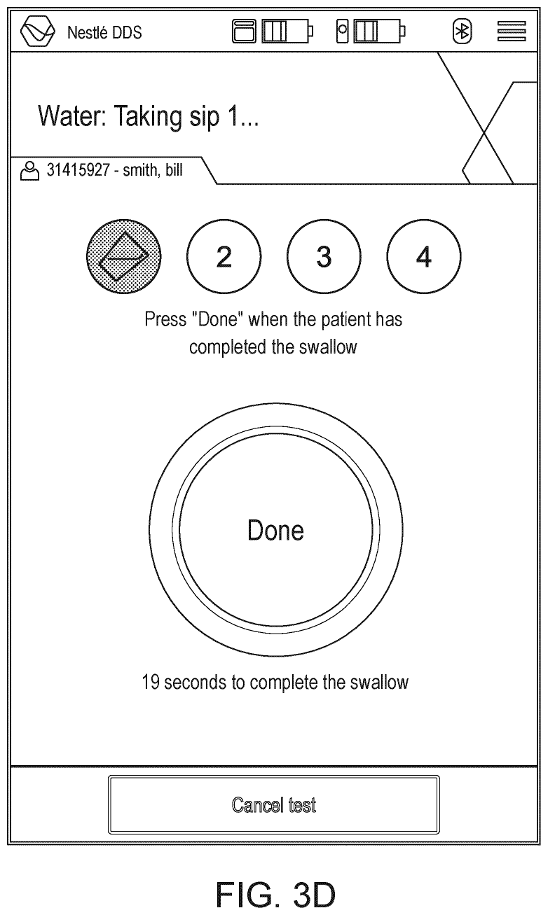

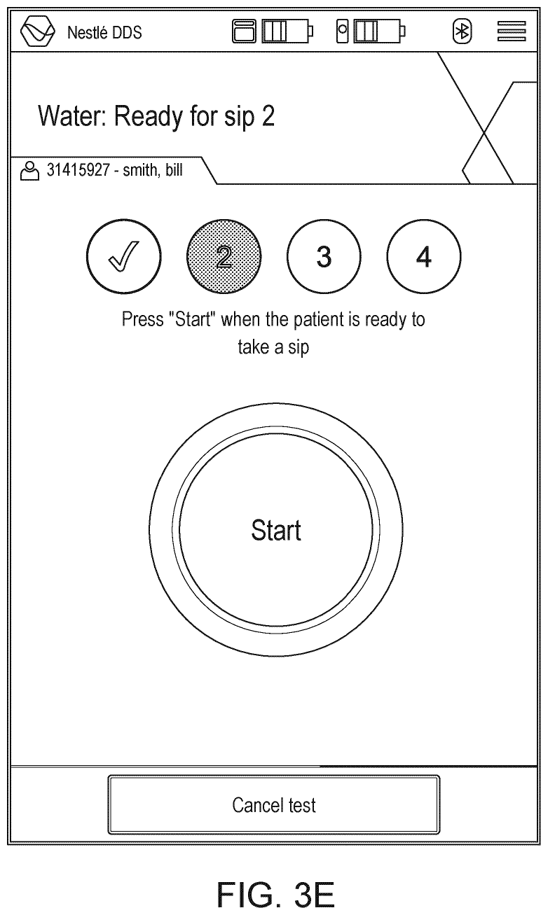

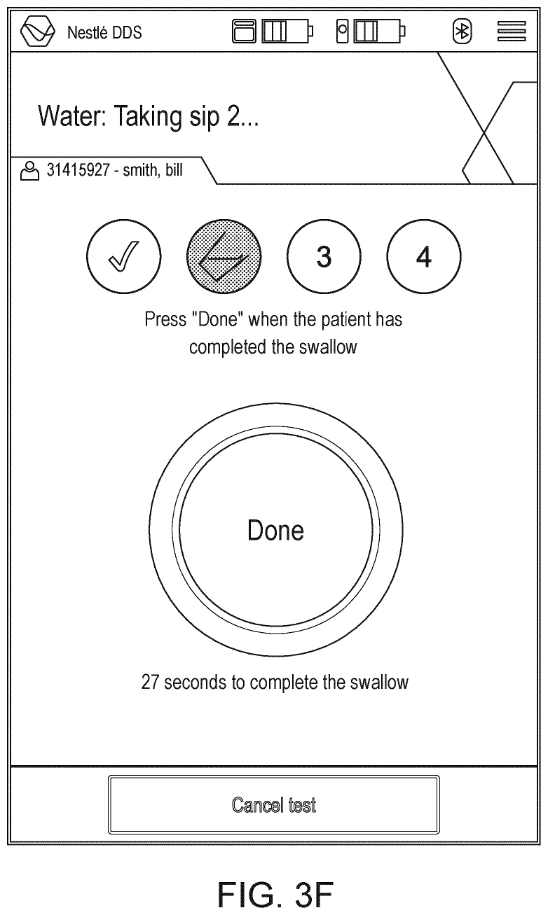

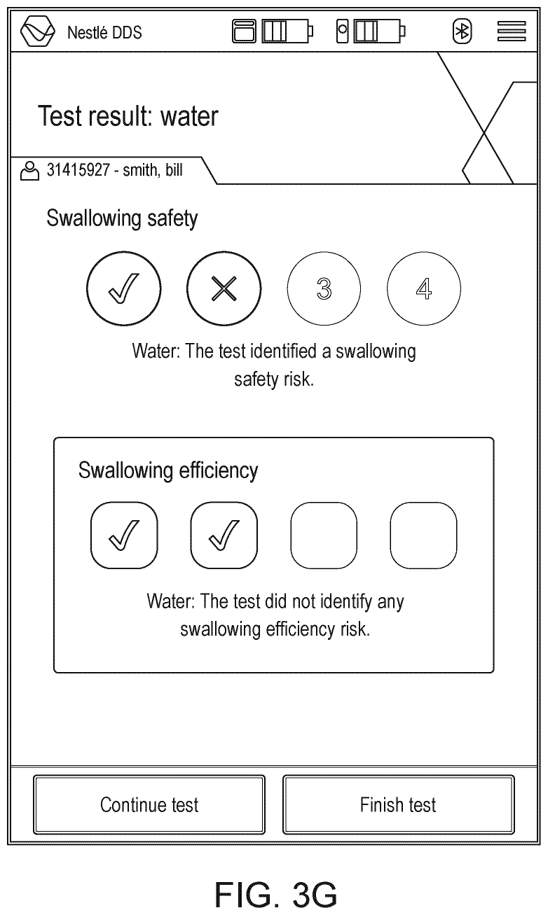

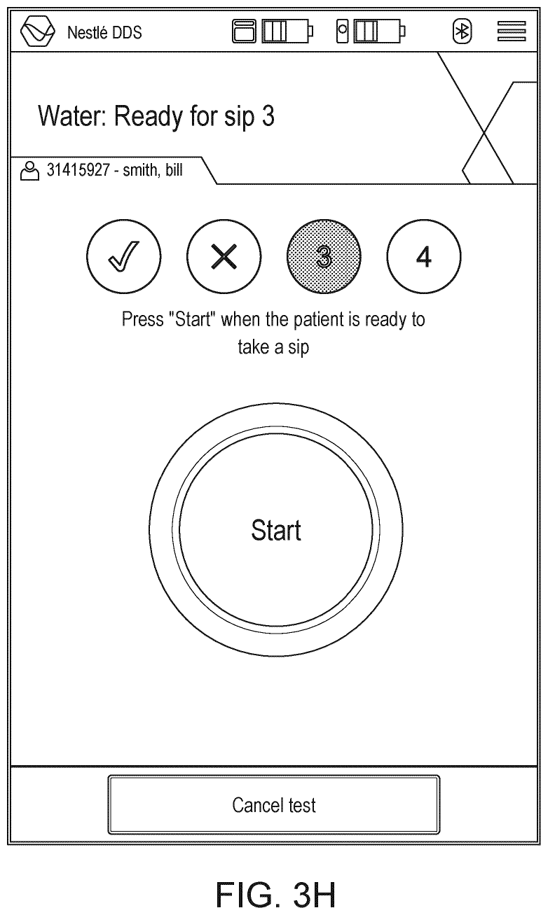

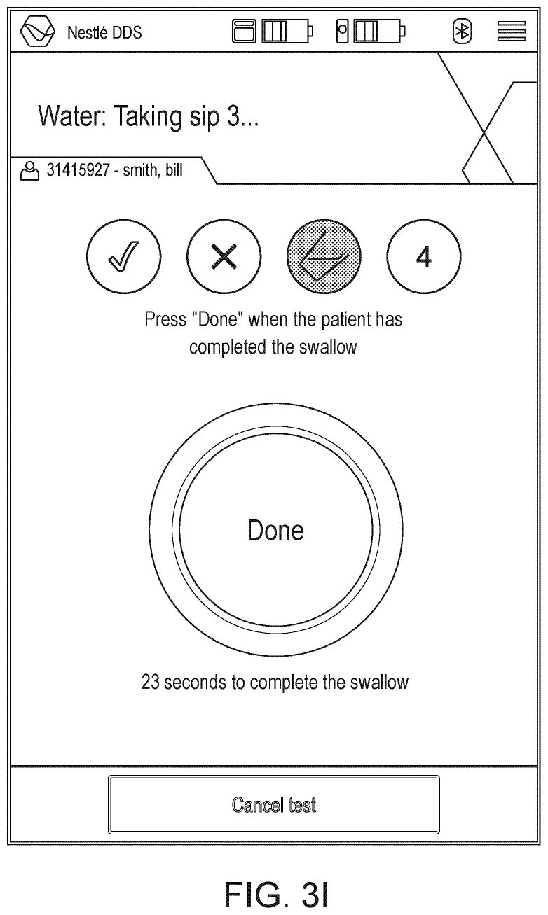

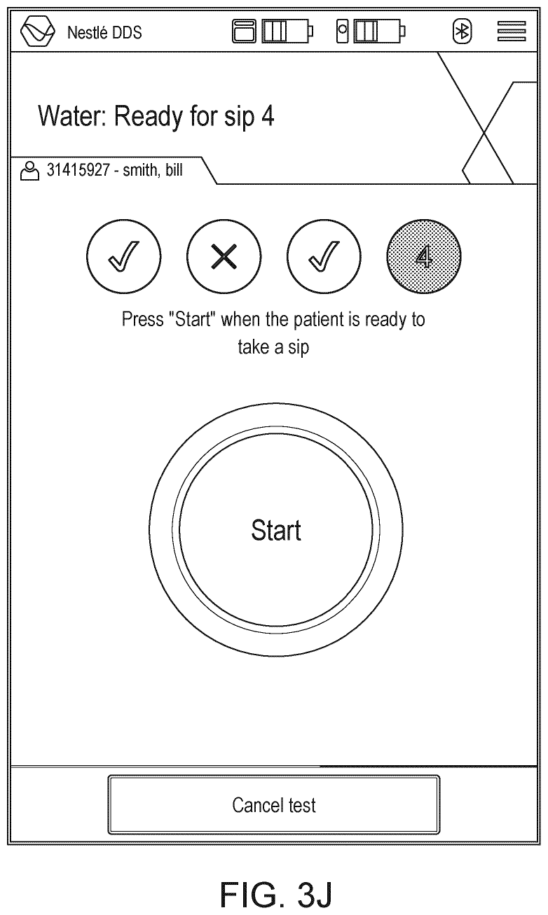

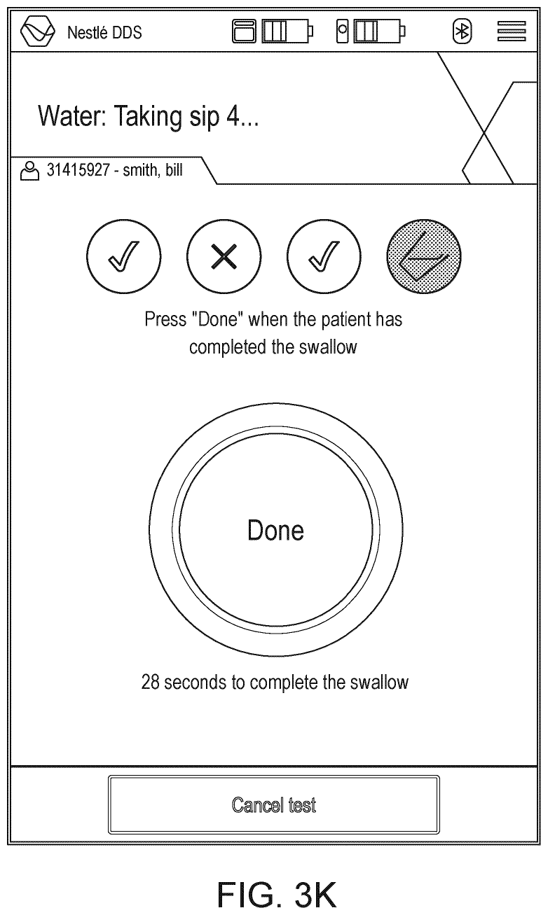

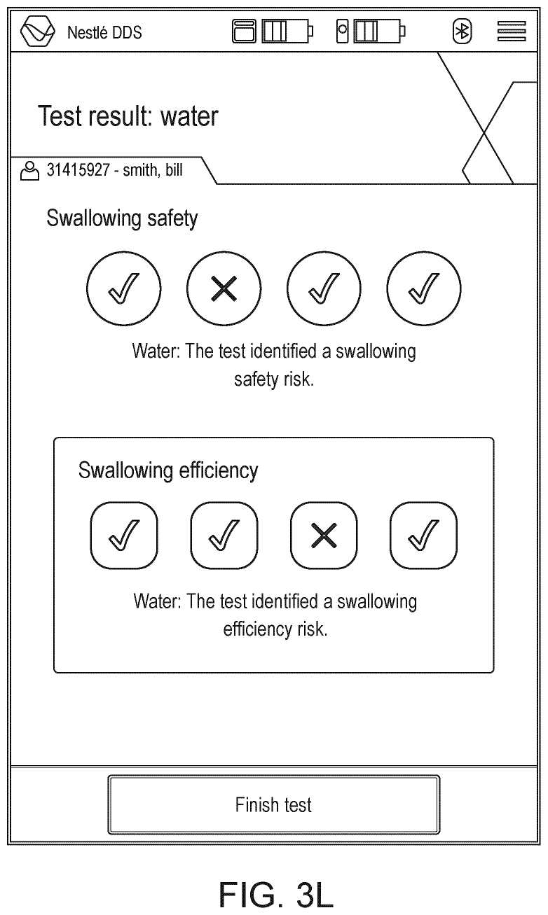

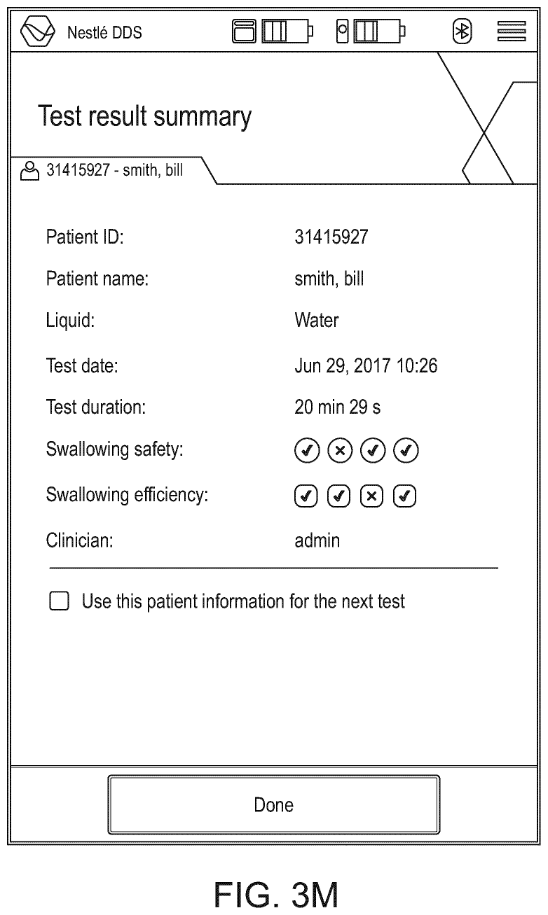

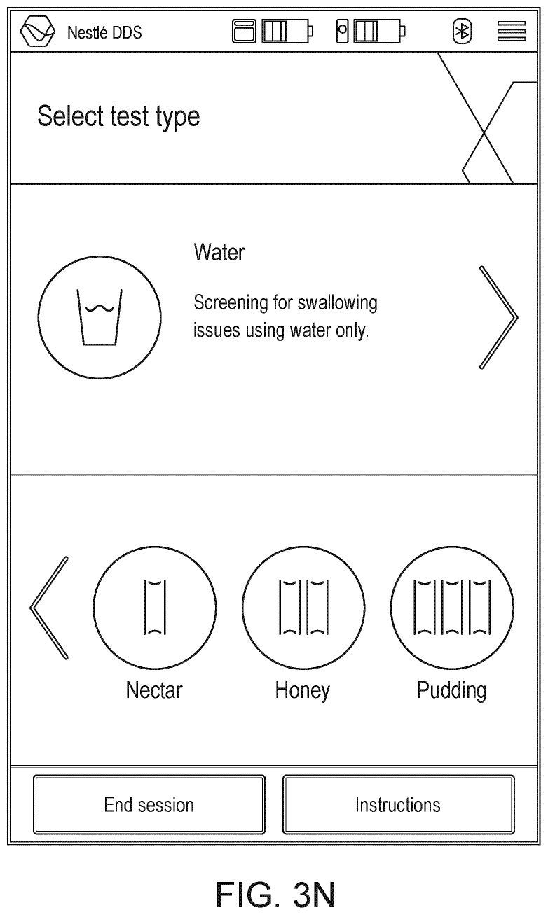

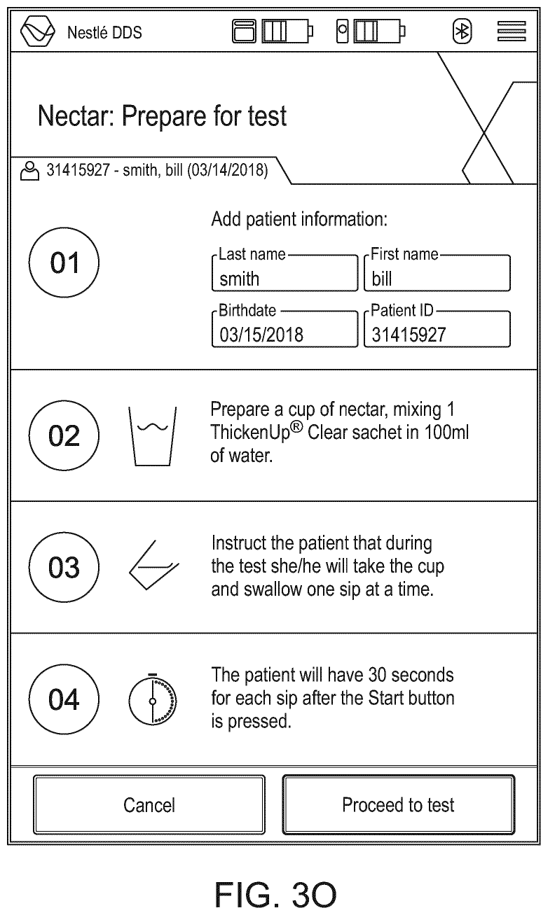

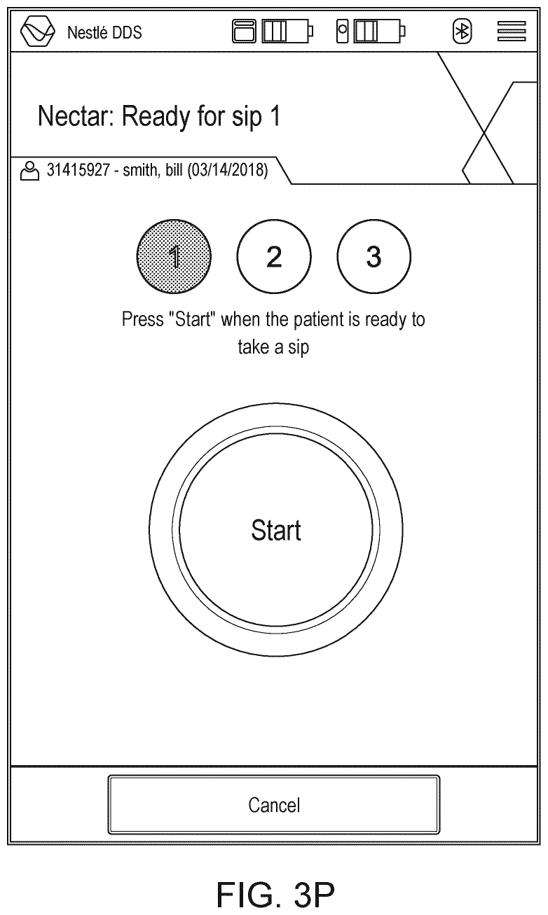

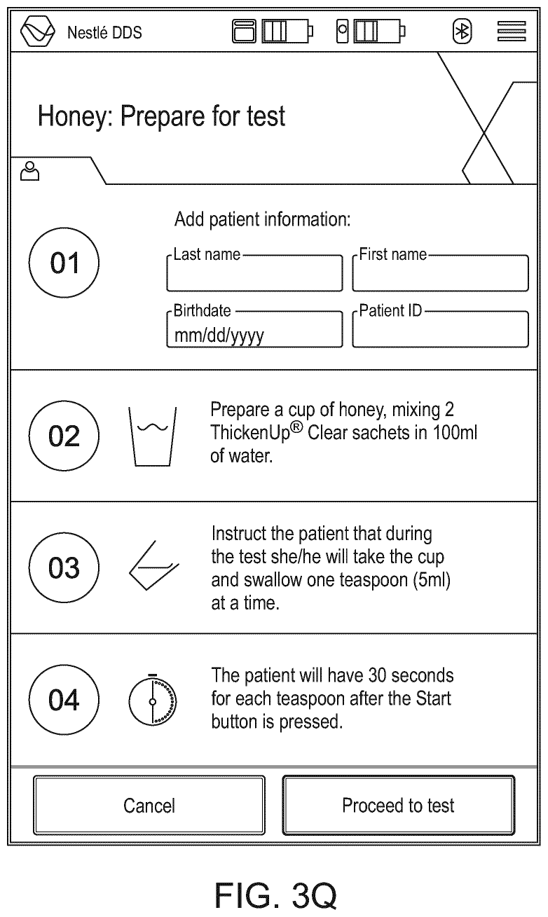

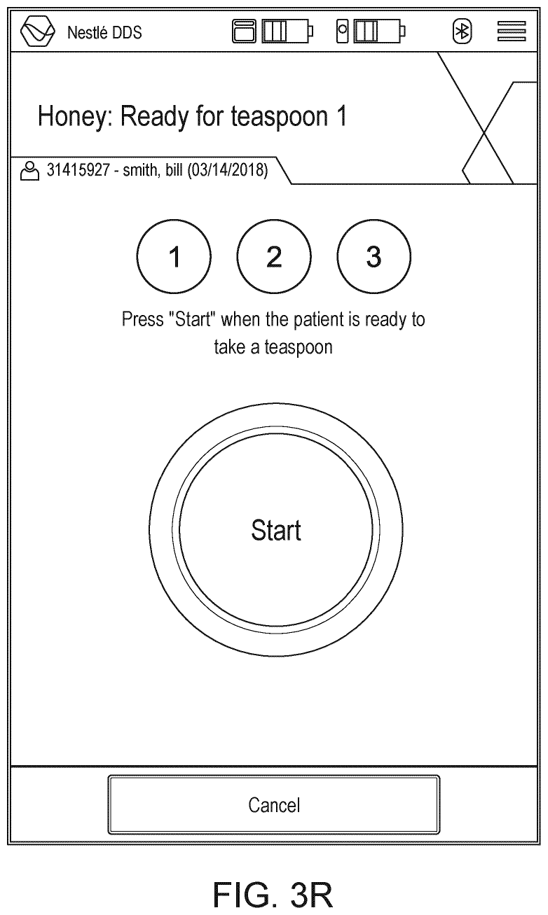

FIGS. 3A-3N generally illustrate non-limiting examples of screens displayed in the first embodiment. The screens are displayed by the device 100, e.g., on the user interface 104, and each bolus is classified as a separate event relative to the other boluses. FIG. 3A generally illustrates that the device 100 can display a plurality of beverage types and can allow a user to identify the type of beverage that will be used in the subsequent screening. This is a point measurement system, i.e., each swallow has no impact on the other swallows. The device 100 can start with the thicker liquid and then to the thin and water, or reverse. Also, a continuous monitoring is possible (e.g., someone may need to receive nectar in the morning, nectar at lunch, and pudding before night). FIG. 3B generally illustrates that the device 100 can respond to selection of water as the beverage type by displaying an interface screen that allows entry of patient information, such as a patient identification number and/or a patient name, and provides instructions for the subsequent screening.

After the user enters input directing the device 100 to begin the screening, the device 100 can display an interface screen for administering the first bolus, as shown in FIG. 3C. The user can provide input indicating that the patient sips the first bolus (e.g., “Start”), and then the device 100 can display an interface screen that allows the user to identify when the patient has completed the swallow if the first bolus (e.g., “Done”), as shown in FIG. 3D. In an embodiment, the user has a time threshold, such as ten minutes or five minutes, to select “Start” otherwise the process has a “timeout,” e.g., pauses or stops. In an embodiment, a time threshold, such as thirty seconds, is given for the user to identify that the patient completed the swallow, and if the time threshold is exceeded, analysis is started automatically.

As shown in FIG. 3E, the classifications of the first bolus can be displayed. In this non-limiting example, analysis of the accelerometry data for the first bolus indicated that the patient did not have a swallowing safety problem or a swallowing efficiency problem for the first bolus, and the device 100 displays these classifications accordingly. The user can provide input indicating that the patient sips the second bolus (e.g., “Start”). Then the device 100 can display an interface screen that allows the user to identify when the patient has completed the swallow of the second bolus (e.g., “Done”), as shown in FIG. 3F. In this non-limiting example, analysis of the accelerometry data for the second bolus indicated that the patient had a swallowing safety problem but not a swallowing efficiency problem for the second bolus, and the device 100 displays these classifications accordingly, as shown in FIG. 3G.

As shown in FIG. 3H, the classifications of the first bolus and the second bolus can be displayed separately, and the user can provide input indicating that the patient sips the third bolus (e.g., “Start”). The user can stop or cancel the test at any time, for example by user input into the screens shown in FIGS. 3G and 3H.

Then the device 100 can display an interface screen that allows the user to identify when the patient has completed the swallow of the third bolus (e.g., “Done”), as shown in FIG. 3I. As shown in FIG. 3J, the classifications of the first bolus, the second bolus and the third bolus can be displayed separately, and the user can provide input indicating that the patient sips the fourth bolus (e.g., “Start”). Then the device 100 can display an interface screen that allows the user to identify when the patient has completed the swallow of the fourth bolus (e.g., “Done”), as shown in FIG. 3K.

As shown in FIG. 3L, the classifications of the first bolus, the second bolus, the third bolus, and the fourth bolus can be displayed separately. As shown in FIG. 3M, a screening summary can be provided.

After the screening is completed for one type of beverage, the beverage selection screen (e.g., FIG. 3A) can be displayed again. If a thickened beverage is selected, then a specific type of thickened beverage can be selected, as shown in FIG. 3N. Then the screening can be repeated for this different type of beverage relative to the beverage of the previous screening, for example by proceeding through one or more of the screens shown in FIGS. 3B-3M. Of course, the instructions displayed are preferably specific to the particular type of beverage, as generally illustrated in the screen for nectar depicted in FIG. 3O (which can be followed by the screen shown in FIG. 3P) and in the screen for honey depicted in FIG. 3Q (which can be followed by the screen shown in FIG. 3R). For example, the instructions generally illustrated in FIG. 3B are for water, but for administration of a thickened beverage, may instead address preparation of the thickened beverage, e.g., how to dilute a powder that forms the thickened beverage (FIGS. 3O and 3Q).

For example, if screening with water provides a red result, then a thickened beverage can be tested. As another example, if screening with water provides a green result, the screening can stop without continuing further, or optionally the screening can continue with a thickened beverage or another water test, thereby providing personalized usage of the measures.

The screens and their content shown in FIGS. 3A-3N are merely for illustrative purposes only. Any number of boluses may be used, and the example embodiment using four boluses is not limiting. In some embodiments, if one of the boluses cannot be analyzed, has low quality for the corresponding accelerometry signal (e.g., clipped or noisy), or does not obtain a swallow, an additional bolus can be employed. For example, if four boluses are administered and one of the boluses cannot be analyzed, has low quality for the corresponding accelerometry signal (e.g., clipped or noisy), or does not obtain a swallow, a fifth bolus can be employed. Moreover, a different number of boluses can be used dependent on the type of beverage selected. For example, analysis of water can employ four boluses, and analysis of a thickened beverage can employ three boluses or four boluses.

The first embodiment of the screening that can be performed by the device 100 can be implemented in the following non-limiting exemplary method. The method can comprise receiving, on the device 100 comprising the processor 106, first accelerometry data for a first plurality of swallowing events executed successively by a first individual. The method can comprise transmitting the first accelerometry data to the device 100 from the sensor 102 (e.g., an accelerometer communicatively connected to the device 100).

In the first embodiment, the method can comprise comparing, on the device 100 (e.g., the processor 106) swallowing data (e.g., at least a portion of the first accelerometry data and/or at least a portion of second accelerometry data derived from the first accelerometry data) against preset classification criteria defined for each of swallowing safety and swallowing efficiency. The method can comprise classifying each of the first plurality of swallowing events with a swallowing safety classification and a swallowing efficiency classification based at least partially on the comparing of the swallowing data against the preset classification criteria, the swallowing safety classification is identified from at least one predetermined swallowing safety classification, and the swallowing efficiency classification is identified from at least one predetermined swallowing efficiency classification.

In the first embodiment, the processor 106 can classify each of the first plurality of swallowing events independently from the other swallowing events to provide independent point measurements for the first plurality of swallowing events, e.g., the analysis of each swallow has no impact on the analysis of the other swallows. The classifying by the processor 106 of each of the first plurality of swallowing events can be real-time relative to the corresponding swallowing event.

In the first embodiment, the method can comprise producing, from the device 100 (e.g., from the user interface 104), one or more first outputs comprising at least one of audio and/or graphics that identify the swallowing safety classification and the swallowing efficiency classification for each of the first plurality of swallowing events. The one or more first outputs identifying of the swallowing safety classification and the swallowing efficiency classification for each of the first plurality of swallowing events can be real-time relative to the corresponding swallowing event.

The device 100 can comprise a housing, and the processor 106 and the user interface 104 each can be positioned within the housing and/or mechanically connected to the housing.

In the first embodiment, the method can comprise accepting user input on the device 100 (e.g., on the user interface 104) user input identifying at least one parameter selected from the group consisting of a type of sensor that provides the first accelerometry data and a type of beverage consumed during the first plurality of swallowing events.

In the first embodiment, the method can comprise producing, from the device 100, one or more second outputs comprising at least one of audio and/or graphics that instruct administration of a plurality of doses of beverage, and the first plurality of swallowing events each correspond to one of the plurality of doses of beverage. For example, the method can comprise instructing administration of a first dose of beverage, then identifying the swallowing safety classification and the swallowing efficiency classification for a first swallowing event corresponding to the first dose of beverage, then instructing administration of a second dose of beverage, and then identifying the swallowing safety classification and the swallowing efficiency classification for a second swallowing event corresponding to the second dose of beverage. The device 100 (e.g., the user interface 104) can identify the swallowing safety classification and the swallowing efficiency classification for the first swallowing event simultaneously relative to each other. The method can comprise, after the identifying of the swallowing safety classification and the swallowing efficiency classification for the second swallowing event, instructing administration of a third dose of beverage, then identifying the swallowing safety classification and the swallowing efficiency classification for a third swallowing event corresponding to the third dose of beverage. The method can comprise, after the identifying of the swallowing safety classification and the swallowing efficiency classification for the third swallowing event, instructing administration of a fourth dose of beverage, then identifying the swallowing safety classification and the swallowing efficiency classification for a fourth swallowing event corresponding to the third dose of beverage.

In the first embodiment, the predetermined swallowing safety classifications can comprise a first swallowing safety classification indicative of a safe event and a second swallowing safety classification indicative of an unsafe event, and the predetermined swallowing efficiency classifications can comprise a first swallowing efficiency classification indicative of an efficient event and a second swallowing efficiency classification indicative of an inefficient event. The one or more first outputs can comprise at least one icon for each of the first plurality of swallowing events, the at least one icon is displayed on the user interface 104 of the device 100, at least a portion of the at least one icon can be a first color for the first swallowing safety classification or a second color different than the first color for the second swallowing safety classification, and at least a portion of the at least one icon can be a third color for the first swallowing efficiency classification or a fourth color different than the third color for the second swallowing efficiency classification.

In the first embodiment, the method can comprise storing the swallowing safety classification and the swallowing efficiency classification for each of the first plurality of swallowing events in the device 100 in a first profile associated with the first individual (e.g., in the memory element 107). The method can further comprise: screening, with the device 100, a second plurality of swallowing events executed by the first individual subsequent to the first plurality of swallowing events, the first plurality of swallowing events executed on a first beverage having a first viscosity, and the second plurality of swallowing events executed on a second beverage having a second viscosity different than the first viscosity. Preferably, the method comprises storing the swallowing safety classification and the swallowing efficiency classification for each of the first plurality of swallowing events on the device 100 (e.g., the memory element 107) in association with identification of the first beverage in the first profile associated with the first individual; and storing the swallowing safety classification and the swallowing efficiency classification for each of the second plurality of swallowing events on the device 100 (e.g., the memory element 107) in association with identification of the second beverage in the first profile associated with the first individual.

In the first embodiment, the method can comprise comparing, on the processor 106, the swallowing safety and efficiency classifications for the first plurality of swallowing events to the swallowing safety and efficiency classifications for the second plurality of swallowing events. The method can comprise: screening, on the device 100, a second plurality of swallowing events executed by a second individual subsequent to the first plurality of swallowing events; and storing the swallowing safety classification and the swallowing efficiency classification for each of the second plurality of swallowing events in the device 100 in a second profile associated with the second individual (e.g., in the memory element 107).

In the first embodiment, the method can comprise: screening, on the device 100, a second plurality of swallowing events executed by the first individual subsequent to the first plurality of swallowing events; and comparing, on the processor 106, the swallowing safety and efficiency classifications for the first plurality of swallowing events to the swallowing safety and efficiency classifications for the second plurality of swallowing events.

The method disclosed above is a non-limiting example, and the first embodiment of the swallowing screening can optionally use one or more of the steps disclosed above, can lack one or more of the steps disclosed above, and/or can use one or more additional steps not disclosed above.

Patient-Level Analysis

In a second and particularly preferred embodiment which is alternative to the first embodiment, the bolus-level data is extrapolated to patient-level data (“rolled-up”). Extrapolation of bolus-level data to a single result for swallowing safety of the patient and a single result for swallowing efficiency of the patient is “patient-level” analysis herein.

In some embodiments, each bolus is given a binary classification, and the rolled-up results are determined based on the number of bolus-level classification results (e.g., a number of safe swallow events, a number of unsafe swallow events, a number of efficient swallow events, or a number of inefficient swallow events). However, in a preferred embodiment, each swallowing event can be assigned a probability or a percentile based on the corresponding extracted meta-features. Although the individual swallowing events can optionally be classified as safe or unsafe and/or as efficient or inefficient, preferably these optional classifications are not displayed by the device in this preferred embodiment.

Instead, the probability or percentile of each swallowing event with respect to safety can be combined with the probabilities or percentiles of the other swallowing events with respect to safety to determine a single safety result for the patient, for example by comparing the geometric mean of the swallowing safety probabilities to a predetermined threshold. The probability or percentile of each swallowing event with respect to efficiency can be combined with the probabilities or percentiles of the other swallowing events with respect to efficiency to determine a single efficiency result for the patient, for example by comparing the geometric mean of the swallowing efficiency probabilities to a predetermined threshold. Accordingly, in the preferred form of the second embodiment, the screening goes from a probabilistic set of results at bolus level to a binary outcome at patient level.

In other words, the second embodiment of the device 100 preferably analyzes the accelerometry data from the first plurality of swallowing events to determine a probability or percentile of each swallowing event with respect to safety and a probability or percentile of each swallowing event with respect to efficiency. After the first plurality of events is completed, the device 100 uses these probabilities or percentiles to determine a single result for swallowing safety of the patient and a single result for swallowing efficiency of the patient. Preferably the probability or percentile of each swallowing event is not displayed to the user, e.g., not displayed on the device 100 or any apparatus in communication with the device 100. As noted above, the individual swallowing events can optionally be classified as safe or unsafe and/or as efficient or inefficient, but preferably these optional classifications are not displayed by the device 100.

The second embodiment of the device 100 can indicate whether each of the first plurality of swallowing events provided a usable signal or not, for example if an accelerometry signal is 1) a missing swallow, 2) clipped from start swallow, 3) clipped from end swallow or 4) noisy signal. Each of these four unusable signals is described as a “grey” signal herein.

Preferably the determinations are automatic, such that minimal user input (if any) is needed for the dual-axis accelerometry signals to be processed and used to determine a probability or percentile of each swallowing event with respect to safety and a probability or percentile of each swallowing event with respect to efficiency and then a single result for swallowing safety of the patient and a single result for swallowing efficiency of the patient. For example, in some embodiments the only user input that is needed is identification of when each bolus has been administered and subsequent identification of when swallowing of each bolus is completed.

In the second embodiment of the device 100, each of the first plurality of swallowing events can be assigned a probability or percentile with respect to swallowing safety independently from the other swallowing events to provide independent point measurements for safety for the first plurality of swallowing events. Each of the first plurality of swallowing events can be assigned a probability or percentile with respect to swallowing efficiency independently from the other swallowing events to provide independent point measurements for efficiency for the first plurality of swallowing events. Preferably, determination by the processor 106 of the probability or percentile with respect to swallowing safety and the probability or percentile with respect to swallowing efficiency for each of the first plurality of swallowing events is real-time relative to the corresponding swallowing event. The probability or percentile for safety and the probability or percentile for efficiency can be stored by the device 100 and/or uploaded to a cloud.

In the second embodiment, the user interface 104 of the device 100 is preferably configured to provide one or more first outputs comprising at least one of audio and/or graphics that identify whether each of the first plurality of swallowing events provided a usable signal or not. Preferably, the one or more first outputs by the user interface 104 are each real-time relative to the corresponding swallowing event. The device 100 can optionally store the signal.

In the second embodiment of the device 100, the processor 106 can be configured to use the user interface 104 to provide one or more second user outputs comprising at least one of audio and/or graphics that instruct administration of a plurality of doses of beverage, and each of the first plurality of swallowing events correspond to one of the plurality of doses of beverage.

For example, the processor 106 can be configured to use the user interface 104 to instruct administration of a first dose of beverage (and optionally subsequently accept user input indicating that swallowing of the first dose is completed). Then the user interface 104 can identify whether a first swallowing event corresponding to the first dose of beverage provided a usable signal or not. Then the user interface 104 can instruct administration of a second dose of beverage (and optionally subsequently accept user input indicating that swallowing of the second dose is completed). Then the user interface 104 can identify whether a second swallowing event corresponding to the second dose of beverage provided a usable signal or not.

In the second embodiment, the processor 106 can be configured to use the user interface 104, after identifying whether the second swallowing event provided a usable signal or not, to instruct administration of a third dose of beverage (and optionally subsequently accept user input indicating that swallowing of the third dose is completed). Then the user interface 104 can identify whether the third swallowing event corresponding to the third dose of beverage provided a usable signal or not. The processor 106 can be configured to use the user interface 104, after identifying whether the third swallowing event provided a usable signal or not, to instruct administration of a fourth dose of beverage (and optionally subsequently accept user input indicating that swallowing of the fourth dose is completed). Then the user interface 104 can identify whether a fourth swallowing event corresponding to the fourth dose of beverage provided a usable signal or not.

In the second embodiment, the one or more first outputs by the device 100 can comprise at least one icon displayed on the user interface 104 for each of the first plurality of swallowing events. At least a portion of the at least one icon can be a first color to indicate a usable signal (e.g., blue) or a second color different than the first color to indicate no signal or an unusable signal (e.g., grey).

As noted above, the second embodiment is particularly preferred and extrapolates the bolus-level data, e.g., the percentile or probability determined for each swallowing event, to patient-level data, e.g., a single safety result and a single efficiency result for the first plurality of swallowing events. Accordingly, the processor 106 can classify the first plurality of swallowing events with a single swallowing safety classification and a single swallowing efficiency classification based at least partially on the percentile or probability determined for each swallowing event.

For example, in the second embodiment of the device 100, the processor 106 may compare the geometric mean of the swallowing safety probabilities to a predetermined threshold. The comparison can be used to identify a single swallowing safety classification from at least one predetermined swallowing safety classification. In the second embodiment of the device 100, the processor 106 may compare the geometric mean of the swallowing efficiency probabilities to a predetermined threshold. The comparison can be used to identify a single swallowing efficiency classification from at least one predetermined swallowing efficiency classification. The processor 106 can provide one or more third outputs identifying the classifications, preferably on the user interface 104 of the device 100. If only one data point is used, the bolus-level result is also the patient-level result.

In the second embodiment of the device 100, the predetermined swallowing safety classifications comprise a first swallowing safety classification indicative of a safe event and a second swallowing safety classification indicative of an unsafe event. The predetermined swallowing efficiency classifications can comprise a first swallowing efficiency classification indicative of an efficient event and a second swallowing efficiency classification indicative of an inefficient event. The one or more third outputs can comprise at least one icon displayed on the user interface 104 after completion of the first plurality of swallowing events. At least a portion of the at least one icon can be a first color for the first swallowing safety classification (e.g., green) or a second color different than the first color for the second swallowing safety classification (e.g., red). At least a portion of the at least one icon can be a third color for the first swallowing efficiency classification (e.g., green) or a fourth color different than the third color for the second swallowing efficiency classification (e.g., red).

In the second embodiment, the memory element 107 can store the single swallowing safety classification and the single swallowing efficiency classification for the first plurality of swallowing events in a first profile associated with the first individual. The device 100 can be used to monitor the first individual by periodically screening the first individual and saving the results of the periodic screenings in the memory element 107. The results from the periodic screenings can be used to modulate the diet; for example, if the swallowing of the patient becomes worse over time, a thicker product may be needed, or the reverse.

For example, the processor 106 can be configured to screen a second plurality of swallowing events executed by the first individual subsequent to the first plurality of swallowing events, for example at least one day later, at least one week later, at least one month later, or at least one year later. The processor 106 can be configured to compare the single swallowing safety classification and the single swallowing efficiency classification for the first plurality of swallowing events to the single swallowing safety classification and the single swallowing efficiency classification for the second plurality of swallowing events, respectively. Such periodic comparisons can be used to monitor the relative progress or decline of a patient.

In the second embodiment, the device 100 can screen the individual for swallowing safety and swallowing efficiency for each of a plurality of beverages, such as one or more of water (50 mPa·s or less, e.g. 1 mPa·s), nectar (51-350 MPa·s), honey (351-1750 mPa·s) or pudding (>1750 mPa·s), and most preferably screen each type of beverage separately (i.e., initially screen one or more boluses of a first beverage, then screen one or more boluses of a second beverage). The device 100 can screen the first individual for one or more types of beverages at a first time and then screen the individual for the one or more types of beverages again periodically thereafter, for example at least one day, at least one week, at least one month, or at least one year between screenings.

For example, the first plurality of swallowing events can be executed on a first beverage having a first viscosity. The processor 106 can be configured to screen a second plurality of swallowing events executed by the first individual subsequent to the first plurality of swallowing events, and the second plurality of swallowing events can be executed on a second beverage having a second viscosity different than the first viscosity. The processor 106 can be configured to store (e.g., in the memory element 107) the single swallowing safety classification and the single swallowing efficiency classification for the first plurality of swallowing events in association with identification of the first beverage in the first profile associated with the first individual. The processor 106 can be configured to store (e.g., in the memory element 107) the single swallowing safety classification and the single swallowing efficiency classification for the second plurality of swallowing events in association with identification of the second beverage in the first profile associated with the first individual. Periodic screenings of the first and second beverages for the first individual can be performed, i.e., at least one day, at least one week, at least one month, or at least one year between screenings, and the results of these multi-beverage screenings can also be stored in the first profile associated with the first individual.

The device 100 can be used to screen and/or monitor a plurality of individuals, e.g., the first individual, a second individual, and optionally additional individuals. Preferably the individuals are screened autonomously (i.e., the screening results are separate for each individual relative to the screening results of the other individuals). Each of the plurality of individuals can have their own profile and, if desired, can be screened the same day as the other individuals.

For example, the processor 106 can be configured to screen a second plurality of swallowing events executed by a second individual different than the first individual subsequent to the first plurality of swallowing events. The device 100 can store (e.g., in the memory element 107) the single swallowing safety classification and the single swallowing efficiency classification for the second plurality of swallowing events in a second profile associated with the second individual. The processor 106 can be configured to compare the swallowing safety and efficiency classifications for the first individual to the swallowing safety and efficiency classifications for the second individual, preferably while recording similarities or differences in characteristics of the individual, such as one or more of age, gender, height, weight and medical condition.

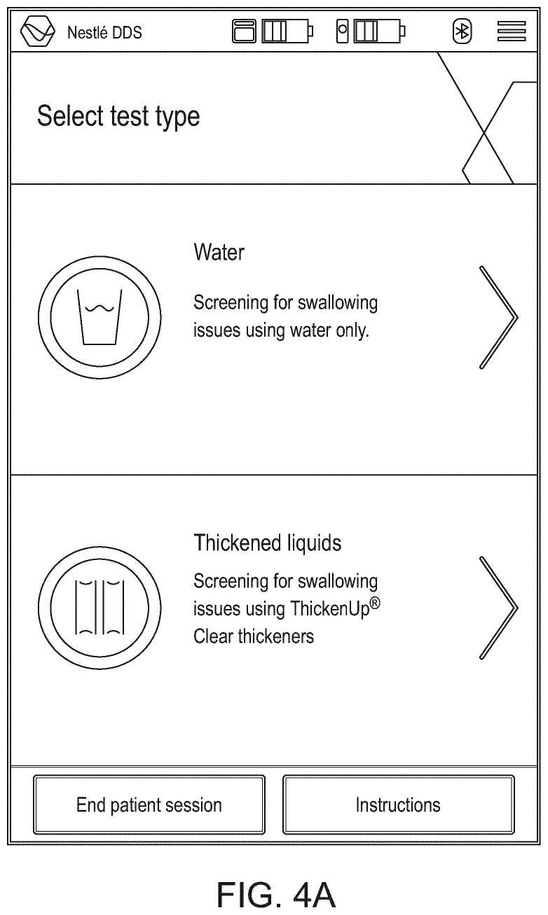

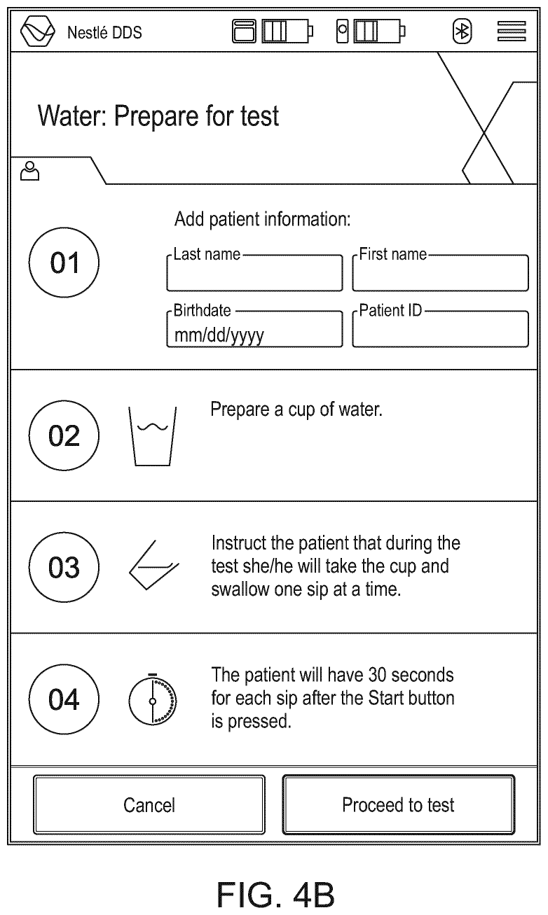

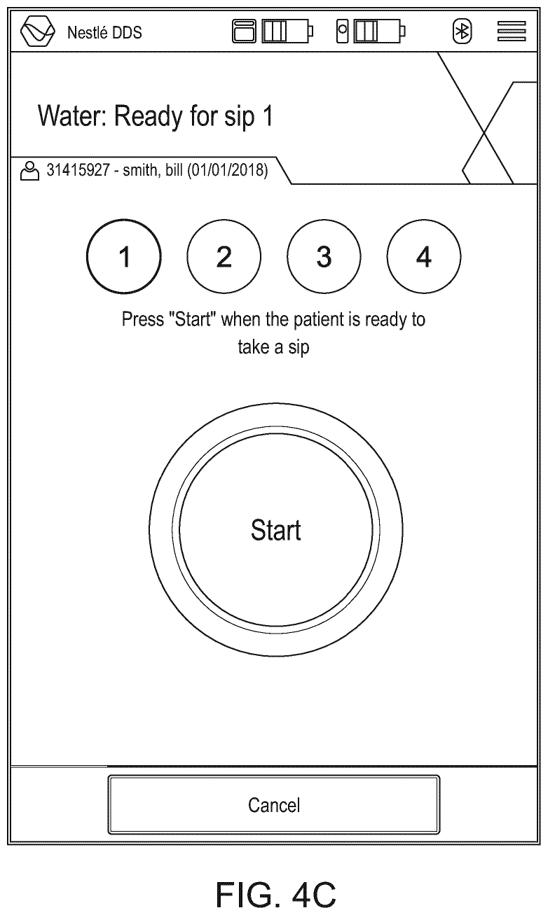

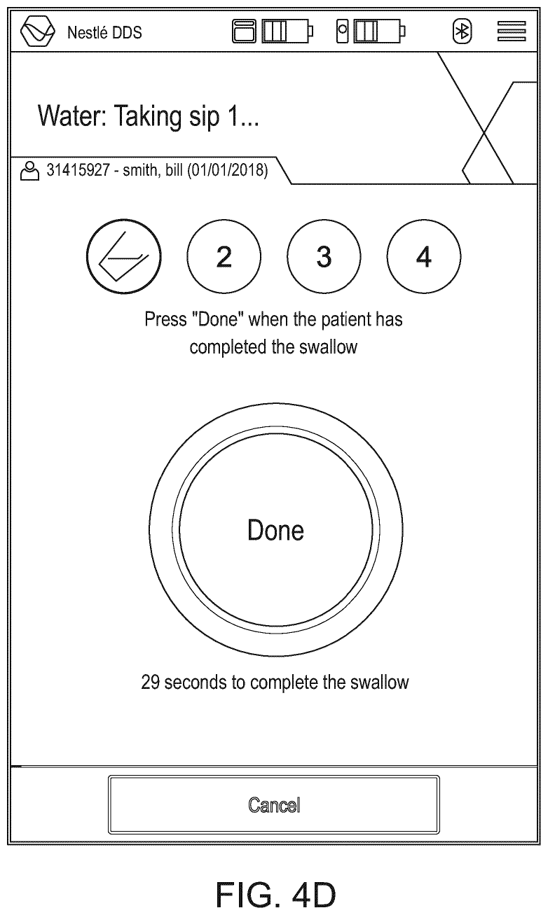

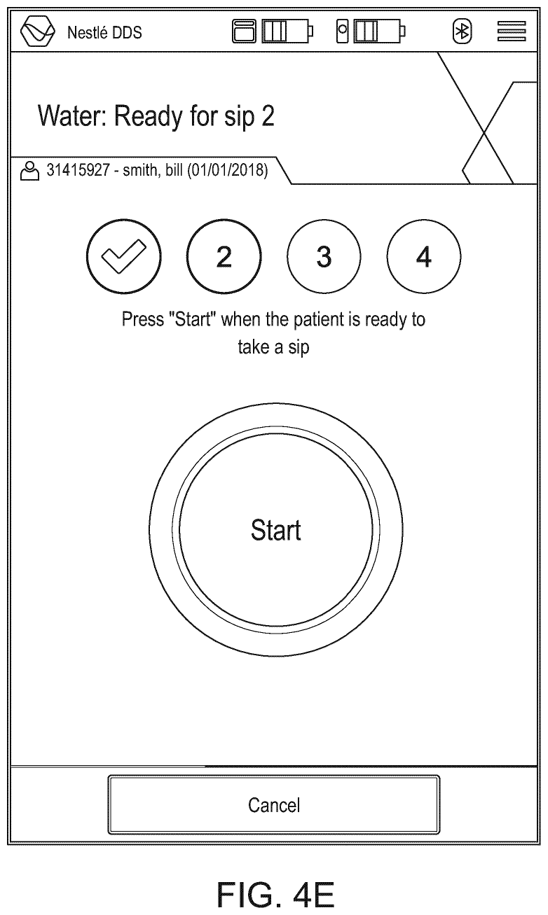

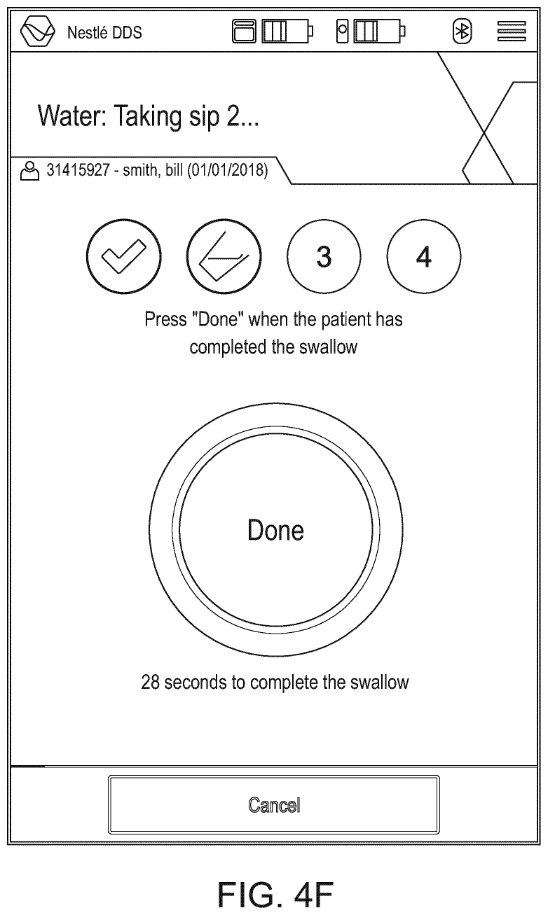

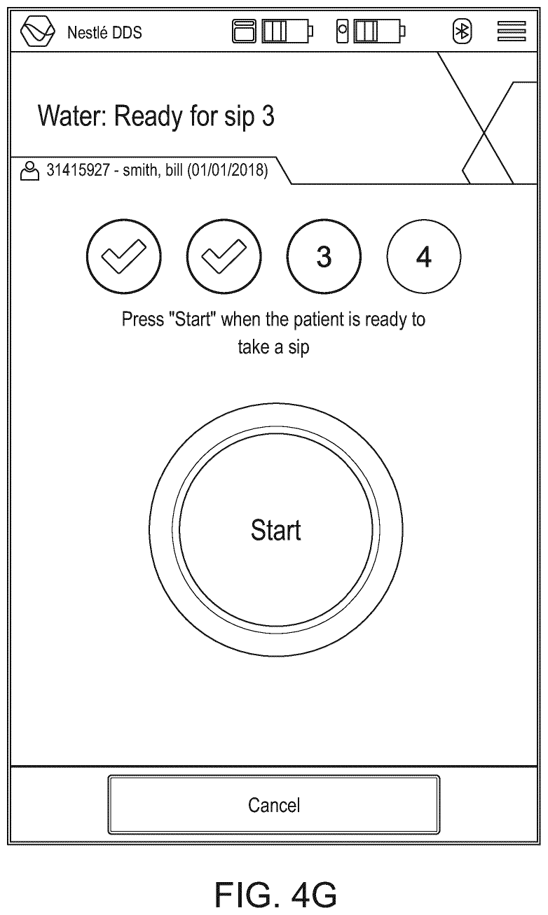

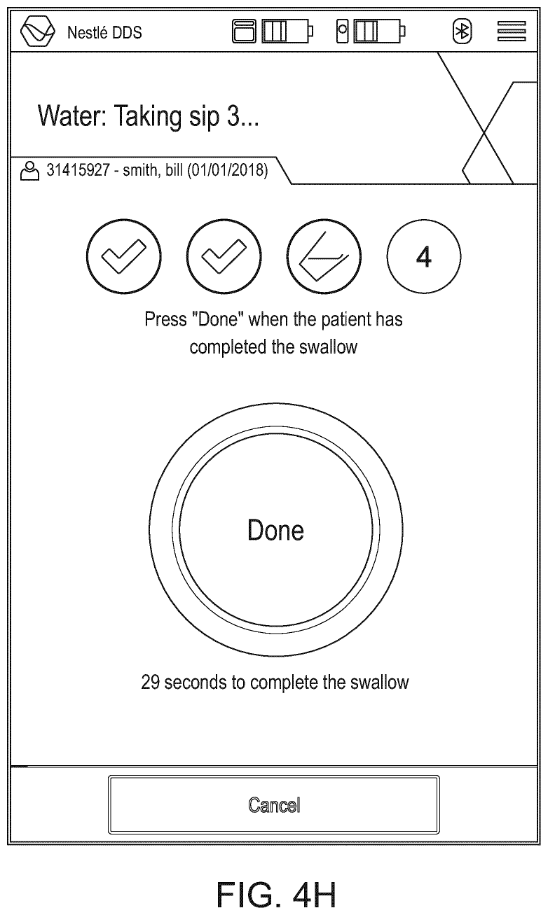

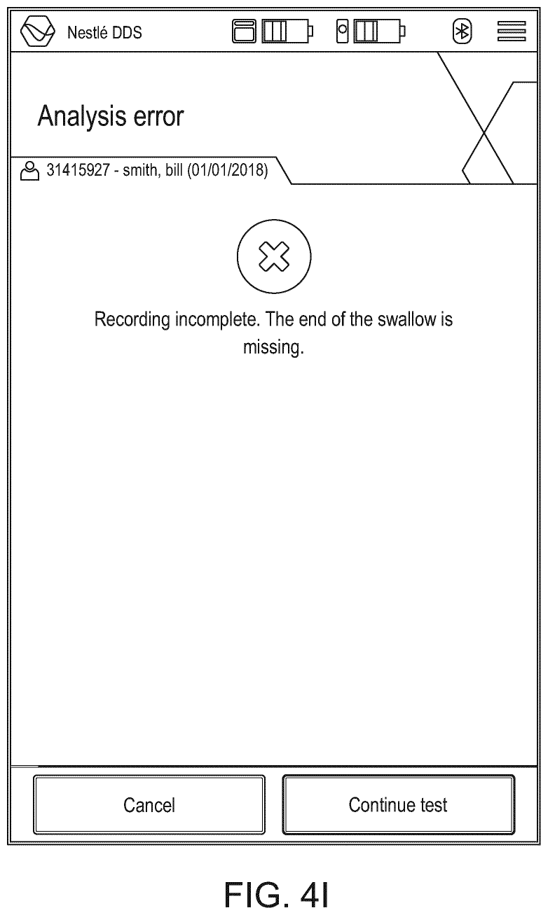

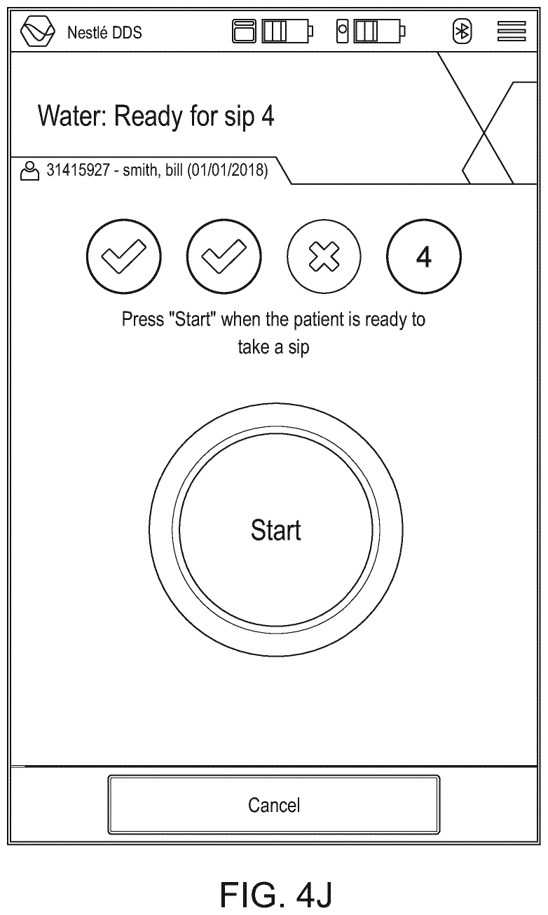

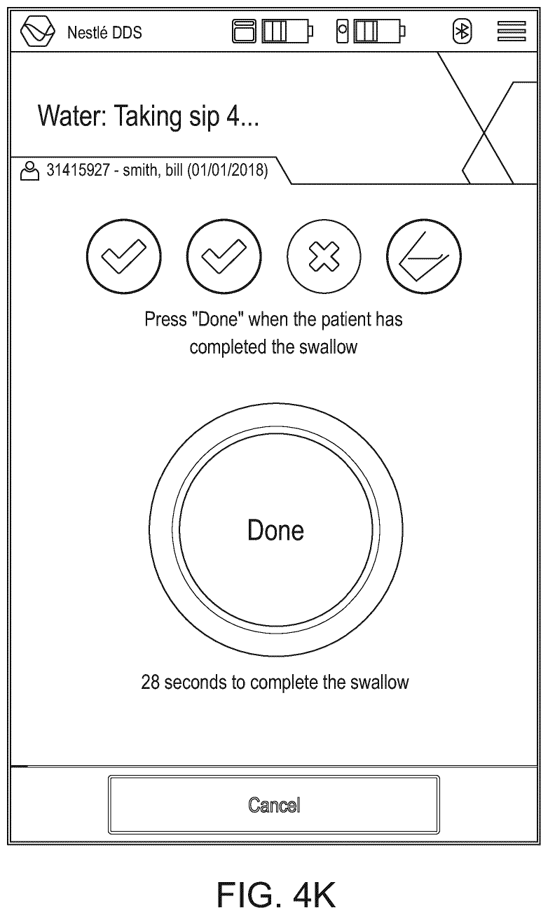

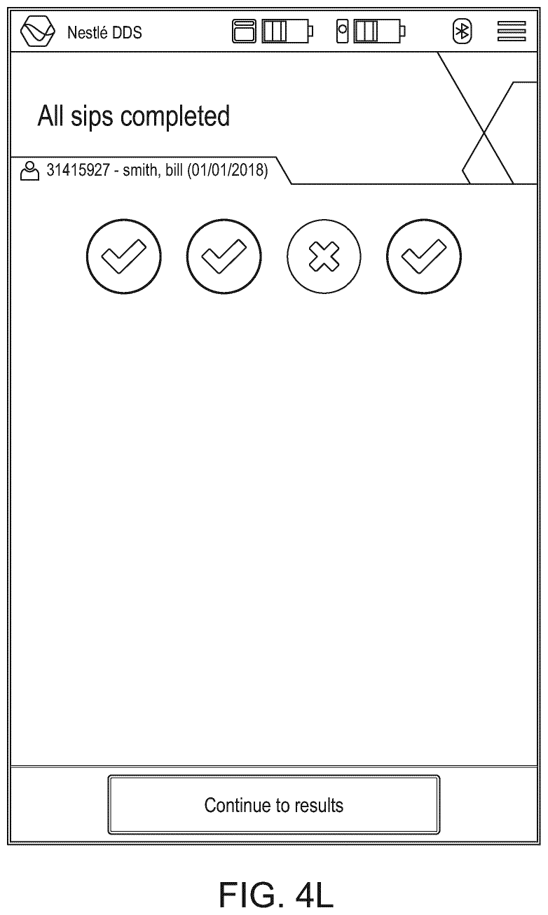

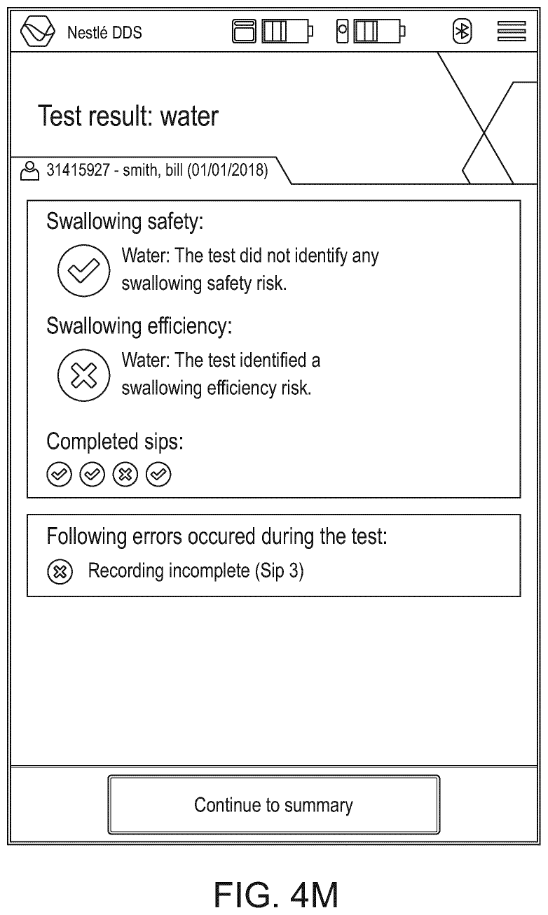

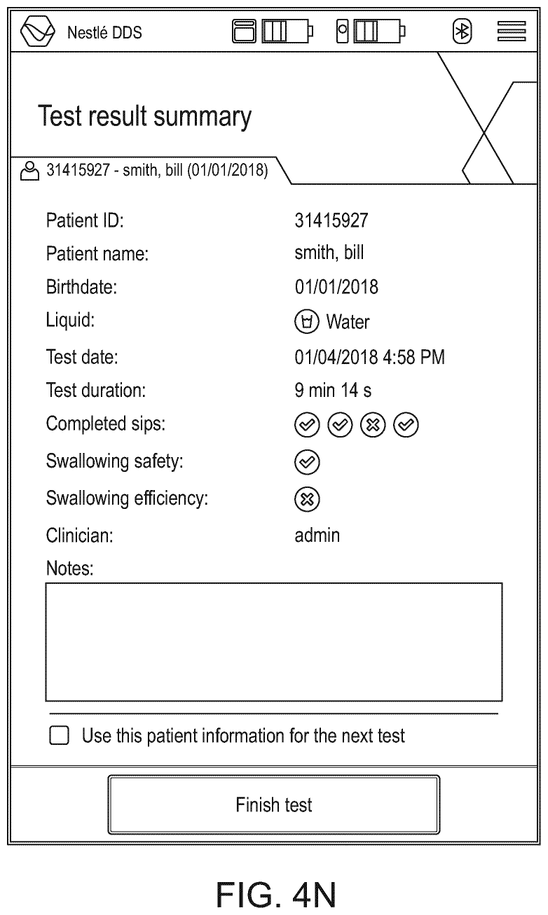

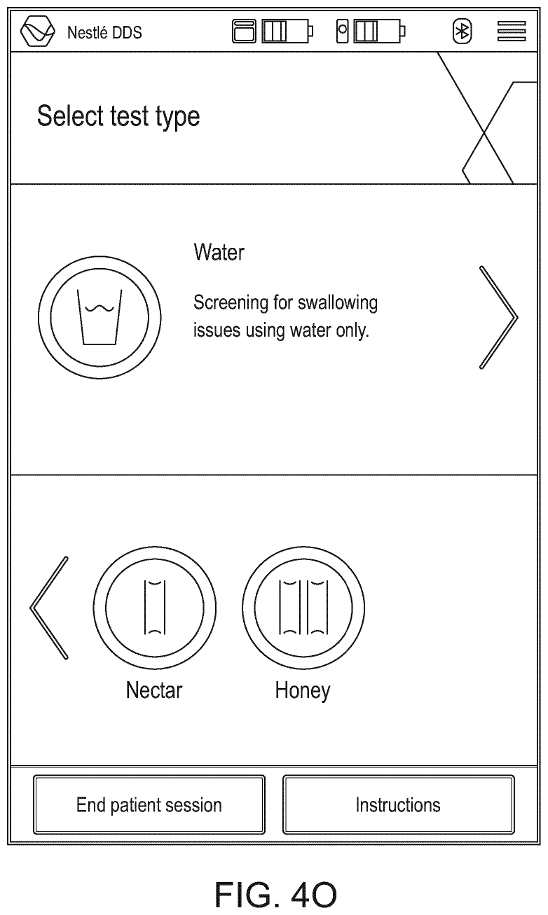

FIGS. 4A-4O generally illustrate non-limiting examples of screens displayed in the second embodiment. The screens are displayed by the device 100, e.g., on the user interface 104, and a probability or percentile for swallowing safety and a probability or percentile for swallowing efficiency is determined for each bolus separately from the other boluses. FIG. 4A generally illustrates that the device 100 can display a plurality of beverage types and can allow a user to identify the type of beverage that will be used in the subsequent screening. FIG. 4B generally illustrates that the device 100 can respond to selection of water as the beverage type by displaying an interface screen that allows entry of patient information, such as a patient identification number, a patient name and/or a patient birthdate, and provides instructions for the subsequent screening.

After the user enters input directing the device 100 to begin the screening, the device 100 can display an interface screen for administering the first bolus, as shown in FIG. 4C. The user can provide input indicating that the patient sips the first bolus (e.g., “Start”), and then the device 100 can display an interface screen that allows the user to identify when the patient has completed the swallow if the first bolus (e.g., “Done”), as shown in FIG. 4D. In an embodiment, a time threshold, such as thirty seconds, is given for the user to identify that the patient completed the swallow, and if the time threshold is exceeded, the accelerometry data associated with the corresponding bolus is not analyzed.

As shown in FIG. 4E, whether the first bolus provided a usable signal can be displayed. In this non-limiting example, the first bolus provided a usable signal, and thus a corresponding output is provided by the user interface 104 (e.g., an icon with a blue color). If the first bolus provided a usable signal, the accelerometry data for the first bolus can be analyzed to determine a probability or percentile for swallowing safety and a probability or percentile for swallowing efficiency, but the device 100 does not display these determinations. The reading can be red or green. The user can provide input indicating that the patient sips the second bolus (e.g., “Start”). Then the device 100 can display an interface screen that allows the user to identify when the patient has completed the swallow of the second bolus (e.g., “Done”), as shown in FIG. 4F.

As shown in FIG. 4G, whether the second bolus provided a usable signal can be displayed. In this non-limiting example, the second bolus provided a usable signal, and thus a corresponding output is provided by the user interface 104 (e.g., an icon with a blue color). If the second bolus provided a usable signal, the accelerometry data for the second bolus can be analyzed to determine a probability or percentile for swallowing safety and a probability or percentile for swallowing efficiency, but the device 100 does not display these determinations. The user can provide input indicating that the patient sips the third bolus (e.g., “Start”). Then the device 100 can display an interface screen that allows the user to identify when the patient has completed the swallow of the third bolus (e.g., “Done”), as shown in FIG. 4H.

As shown in FIG. 4I, whether the third bolus provided a usable signal can be displayed. In this non-limiting example, the third bolus did not provide a usable signal; and thus a corresponding output is provided by the user interface 104 (e.g., a grey icon with an “X” symbol), and the error can be identified (e.g., no sip detected, a missing end or start of swallow, or excessive signal noise). If the third bolus provided a usable signal, the accelerometry data for the third bolus can be analyzed to determine a probability or percentile for swallowing safety and a probability or percentile for swallowing efficiency, but the device 100 does not display these determinations.

Then the device 100 can display an interface screen that allows the user to provide input indicating that the patient sips the fourth bolus (e.g., “Start”), as shown in FIG. 4J. Then the device 100 can display an interface screen that allows the user to identify when the patient has completed the swallow of the fourth bolus (e.g., “Done”), as shown in FIG. 4K. As shown in FIG. 4L, whether the fourth bolus provided a usable signal can be displayed. In this non-limiting example, the fourth bolus provided a usable signal, and thus a corresponding output is provided by the user interface 104 (e.g., an icon with a blue color). If the fourth bolus provided a usable signal, the accelerometry data for the second bolus can be analyzed to determine a probability or percentile for swallowing safety and a probability or percentile for swallowing efficiency, but the device 100 does not display these determinations. If one of the four boluses did not provide a usable signal, preferably the bolus is not repeated, but rather the classifications are made based on the other three boluses.

The unusable signal, herein described as a “grey” signal, has no impact on the outcome. The grey signal does not mean that the result will be green or red and instead just means that there is an unusable measurement. If three grey signals and one usable signal are received, the bolus level analysis will be equivalent to a patient level analysis. For example for water, when four swallows are used and one or more of them provide a grey signal (e.g., the third swallow), the one or more swallows providing a grey signal are excluded (FIG. 4L). The remaining sips have an individual probability at bolus level to be red or green and when rolled up at patient level, the device 100 will return a binary result of red or green as per the roll-up rules embedded in the algorithm.

Preferably, when a grey signal is received, the device 100 does not repeat the measurement and instead proceeds directly to the next measurement. For example, if the third bolus provides a grey signal, the device 100 preferably does not repeat the third bolus and instead proceeds directly to the fourth bolus.

As shown in FIG. 4M, the single swallowing safety classification and the single swallowing efficiency classification can be displayed. As noted above, the device calculates these single classifications based at least partially on the probabilities or percentiles of each of the swallowing events. Any errors can also be identified. As shown in FIG. 4N, a screening summary can be provided.

After the screening is completed for one type of beverage, the beverage selection screen (e.g., FIG. 4A) can be displayed again. If a thickened beverage is selected, then a specific type of thickened beverage can be selected, as shown in FIG. 4O. Then the screening can be repeated for this different type of beverage relative to the beverage of the previous screening, for example by proceeding through one or more of the screens shown in FIGS. 4B-4N. Of course, the instructions displayed are preferably specific to the particular type of beverage. For example, the instructions generally illustrated in FIG. 4B are for water, but for administration of a thickened beverage, may instead address preparation of the thickened beverage, e.g., how to dilute a powder that forms the thickened beverage.

The screens and their content shown in FIGS. 4A-4O are merely for illustrative purposes only. Any number of boluses may be used, and the example embodiment using four boluses is not limiting. Moreover, a different number of boluses can be used dependent on the type of beverage selected. For example, analysis of water can employ four boluses, and analysis of a thickened beverage can employ three boluses.

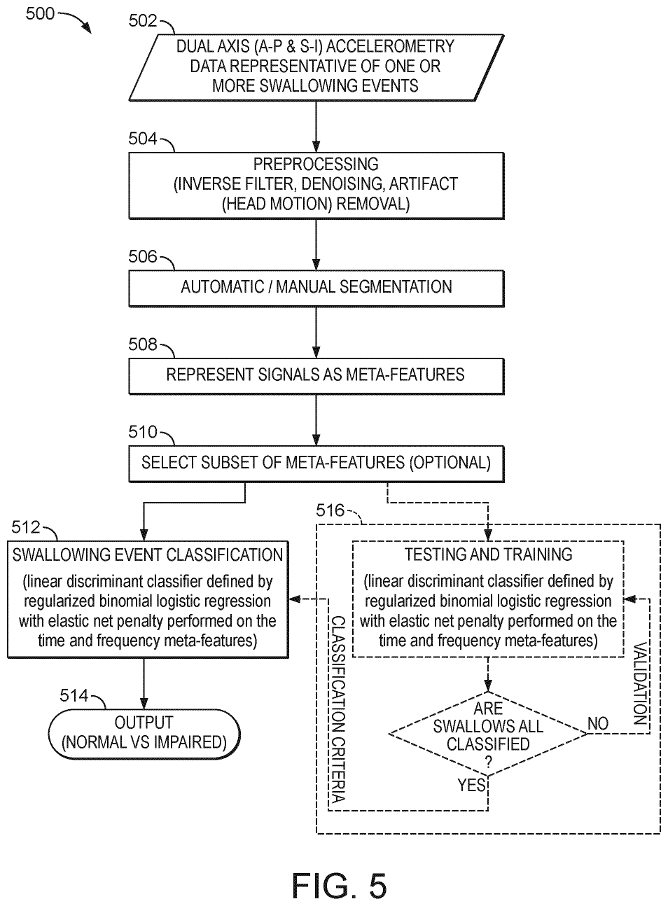

FIG. 5 illustrates a non-limiting example of a method 500 for the second embodiment of the swallowing screening device 100 disclosed above. At Step 502, dual-axis accelerometry data for both the S-I axis and the A-P axis is acquired or provided for one or more swallowing events, for example dual-axis accelerometry data from the sensor 102. Using accelerometry, one can integrate the signal to derive the velocity and similarly one can integrate the velocity to derive the displacement. Nevertheless, other types of sensors can be implemented additionally or alternatively.

At Step 504, the second embodiment of the swallowing screening device 100 can optionally process the dual-axis accelerometry data to condition the accelerometry data and thus facilitate further processing thereof. For example, the dual-axis accelerometry data may be filtered, denoised, and/or processed for signal artifact removal (“preprocessed data”). In an embodiment, the dual-axis accelerometry data is subjected to an inverse filter, which may include various low-pass, band-pass and/or high-pass filters, followed by signal amplification. A denoising subroutine can then applied to the inverse filtered data, preferably processing signal wavelets and iterating to find a minimum mean square error.

The preprocessing may comprise a subroutine for the removal of movement artifacts from the data, for example, in relation to head movement by the patient. Additionally or alternatively, other signal artifacts, such as vocalization and blood flow, may be removed from the dual-axis accelerometry data. Nevertheless, the method 500 is not limited to a specific embodiment of the preprocessing of the accelerometry data, and the preprocessing may comprise any known method for filtering, denoising and/or removing signal artifacts.

At Step 506, the second embodiment of the swallowing screening device 100 can automatically or manually segment the accelerometry data (either raw or preprocessed) into distinct swallowing events. Preferably the accelerometry data is automatically segmented. Additionally or alternatively, manual segmentation may be applied, for example by visual inspection of the data. The method 500 is not limited to a specific process of segmentation, and the process of segmentation can be any segmentation process known to one skilled in this art.

At Step 508, the second embodiment of the swallowing screening device 100 can perform meta-feature based representation of the accelerometry data. For example, one or more time-frequency domain features can be calculated for each axis-specific data set. Combinations of extracted features may be considered herein without departing from the general scope and nature of the present disclosure. Preferably different features are extracted for each axis-specific data set, but in some embodiments the same features may be extracted in each case. Furthermore, other features may be considered for feature extraction, for example, including one or more time, frequency and/or time-frequency domain features (e.g., mean, variance, center frequency, etc.).

At Step 510 (which is optional), the second embodiment of the swallowing screening device 100 may select a subset of the meta-features, preferably based on the previous analysis of similar extracted feature sets derived during classifier training and/or calibration. For example, the most prominent features or feature components/levels extracted from the classifier training data set can be retained as most likely to provide classifiable results when applied to new test data, and are thus selected to define a reduced feature set for training the classifier and ultimately enabling classification. For instance, in the context of wavelet decompositions or other such signal decompositions, techniques such as linear discriminant analysis, principle component analysis, or other such techniques effectively implemented to qualify a quantity and/or quality of information available from a given decomposition level may be used on the training data set to preselect feature components or levels most likely to provide the highest level of usable information in classifying newly acquired signals. Such preselected feature components/levels can then be used to train the classifier for subsequent classifications. Ultimately, these preselected features can be used in characterizing the classification criteria for subsequent classifications.

Accordingly, where the device has been configured to operate from a reduced feature set, such as described above, this reduced feature set can be characterized by a predefined feature subset or feature reduction criteria that resulted from the previous implementation of a feature reduction technique on the classifier training data set. Newly acquired data can thus proceed through the various pre-processing and segmentation steps described above (Steps 504, 506), the various swallowing events so identified then processed for feature extraction at Step 508 (e.g., full feature set), and those features corresponding with the preselected subset retained at step 510 for classification at step 512.

While the above exemplary approach contemplates a discrete selection of the most prominent features, other techniques may also readily apply. For example, in some embodiments, the results of the feature reduction process may rather be manifested in a weighted series or vector for association with the extracted feature set in assigning a particular weight or level of significance to each extracted feature component or level during the classification process. In particular, selection of the most prominent feature components to be used for classification can be implemented via linear discriminant analysis (LDA) (and/or another analysis) on the classifier training data set. Consequently, feature extraction and reduction can be effectively used to distinguish safe swallows from potentially unsafe swallows, and efficient swallows from potentially inefficient swallows. In this regard, the extraction of the selected features from new test data can be compared to preset classification criteria established as a function of these same selected features as previously extracted and reduced from an adequate training data set, to classify the new test data as representative of safe swallows vs. unsafe swallows and/or efficient swallows vs. inefficient swallows. As will be appreciated by the skilled artisan, other feature sets such as frequency, time and/or time-frequency domain features may be used.

At Step 512, the second embodiment of the screening device compares extracted features (or a reduced/weighted subset thereof) of acquired swallow-specific data to preset classification criteria to assign a swallowing safety probability or percentile and a swallowing efficiency probability or percentile to each swallowing event, although these determinations are not displayed.

The method 500 can optionally comprise a training/validation subroutine Step 516 in which a data set representative of multiple swallows is processed such that each swallow-specific data set ultimately experiences the preprocessing, feature extraction and feature reduction disclosed herein. Preferably this step is merely used to configure the device 100 and is not performed when the device 100 is implemented by the commercial user of the device 100.

In this optional step, a validation loop can be applied to the discriminant analysis-based classifier using a cross-validation test. After all events have been classified and validated, output criteria may be generated for future classification without necessarily applying further validation to the classification criteria. Alternatively, routine validation may be implemented to either refine the statistical significance of classification criteria, or again as a measure to accommodate specific equipment and/or protocol changes (e.g. recalibration of specific equipment, for example, upon replacing the accelerometer with same or different accelerometer type/model, changing operating conditions, new processing modules such as further preprocessing subroutines, artifact removal, additional feature extraction/reduction, etc.).

At Step 514, the swallowing safety probabilities/percentiles of each swallowing event can be processed together to calculate and output a single swallowing safety determination. The swallowing efficiency probabilities/percentiles of each swallowing event are processed together to calculate and output a single swallowing efficiency determination.

For example, the user interface 104 of the device 100 can comprise a display that identifies the aggregate swallowing events as indicative of safe swallowing or unsafe swallowing and identifies the aggregate swallowing events as indicative of efficient swallowing or inefficient swallowing. The display can use images such as text, icons, colors, lights turned on and off, and the like. Alternatively or additionally, the user interface 104 can comprise a speaker that uses auditory signals. The present disclosure is not limited to a specific embodiment of the output, and the output can be any means by which the user interface 104 identifies the single swallowing safety classification and the single swallowing efficiency classification to a user of the device 100, such as a clinician or a patient.

The output may then be provided to a clinician who can determine, for example, appropriate treatment, further testing, and/or proposed dietary or other related restrictions. For example, a clinician can adjust feedings by changing consistency or type of food and/or the size and/or frequency of mouthfuls offered to the patient. In this regard, a clinician can determine an acceptable beverage type for the individual if a particular beverage type provided better swallowing safety and/or better swallowing efficiency relative to other beverage types (e.g., an acceptable beverage type can be one or more of water, nectar, honey, or pudding).

Alternative types of vibration sensors other than accelerometers can be used with appropriate modifications to be the sensor 102. For example, a sensor can measure displacement (e.g., a microphone), while the processor 106 records displacement signals over time. As another example, a sensor can measure velocity, while the processor 106 records velocity signals over time. Such signals can then be converted into acceleration signals and processed as disclosed herein and/or by other techniques of feature extraction and classification appropriate for the type of received signal.

In the second embodiment of the device 100, the method 500 can comprise receiving, on the device 100 comprising the processor 106, first accelerometry data for a first plurality of swallowing events executed successively by a first individual. The method can comprise transmitting the first accelerometry data to the device 100 from the sensor 102 (e.g., an accelerometer communicatively connected to the device 100).

In the second embodiment of the device 100, the method 500 can comprise comparing, on the device 100, swallowing data (e.g., at least a portion of the first accelerometry data and/or at least a portion of second accelerometry data derived from the first accelerometry data) against preset classification criteria defined for each of swallowing safety and swallowing efficiency. The method 500 can comprise assigning a swallowing safety probability or percentile to each of the first plurality of swallowing events based at least partially on the comparing of the swallowing data against the preset classification criteria. The method 500 can comprise assigning a swallowing efficiency probability or percentile to each of the first plurality of swallowing events based at least partially on the comparing of the swallowing data against the preset classification criteria.

The second embodiment of the device 100 preferably classifies each of the first plurality of swallowing events independently from the other swallowing events to provide independent point measurements for the first plurality of swallowing events. The classifying by the device 100 of each of the first plurality of swallowing events can be real-time relative to the corresponding swallowing event.

The method 500 can comprise classifying each of the first plurality of swallowing events with a swallowing safety classification and a swallowing efficiency classification based at least partially on the comparing of the swallowing data against the preset classification criteria, the swallowing safety classification is identified from at least one predetermined swallowing safety classification, and the swallowing efficiency classification is identified from at least one predetermined swallowing efficiency classification.

In a preferred embodiment, the method 500 comprises producing, from the device 100 (e.g., from the user interface 104), one or more first outputs comprising at least one of audio and/or graphics that identify the swallowing safety classification and the swallowing efficiency classification for each of the first plurality of swallowing events. The one or more first outputs identifying of the swallowing safety classification and the swallowing efficiency classification for each of the first plurality of swallowing events can be real-time relative to the corresponding swallowing event.

In an embodiment, the device comprises a housing, and the processor 106 and the user interface 104 each are positioned within the housing and/or mechanically connected to the housing.

The method 500 can comprise accepting user input on the device 100 (e.g., on the user interface 104) user input identifying at least one parameter selected from the group consisting of a type of sensor that provides the first accelerometry data and a type of beverage consumed during the first plurality of swallowing events.

In an embodiment, the method 500 comprises producing, from the device 100, one or more second outputs comprising at least one of audio and/or graphics that instruct administration of a plurality of doses of beverage, and the first plurality of swallowing events each correspond to one of the plurality of doses of beverage. For example, the method 500 can comprise instructing administration of a first dose of beverage, then identifying the swallowing safety classification and the swallowing efficiency classification for a first swallowing event corresponding to the first dose of beverage, then instructing administration of a second dose of beverage, and then identifying the swallowing safety classification and the swallowing efficiency classification for a second swallowing event corresponding to the second dose of beverage. The device 100 (e.g., the user interface 104) can identify the swallowing safety classification and the swallowing efficiency classification for the first swallowing event simultaneously relative to each other. The method 500 can comprise, after the identifying of the swallowing safety classification and the swallowing efficiency classification for the second swallowing event, instructing administration of a third dose of beverage, then identifying the swallowing safety classification and the swallowing efficiency classification for a third swallowing event corresponding to the third dose of beverage. The method 500 can comprise, after the identifying of the swallowing safety classification and the swallowing efficiency classification for the third swallowing event, instructing administration of a fourth dose of beverage, then identifying the swallowing safety classification and the swallowing efficiency classification for a fourth swallowing event corresponding to the third dose of beverage.

In an embodiment, the predetermined swallowing safety classifications comprise a first swallowing safety classification indicative of a safe event and a second swallowing safety classification indicative of an unsafe event, and the predetermined swallowing efficiency classifications comprise a first swallowing efficiency classification indicative of an efficient event and a second swallowing efficiency classification indicative of an inefficient event. The one or more first outputs can comprise at least one icon for each of the first plurality of swallowing events, the at least one icon is displayed on the user interface 104 of the device 100, at least a portion of the at least one icon can be a first color for the first swallowing safety classification or a second color different than the first color for the second swallowing safety classification, and at least a portion of the at least one icon can be a third color for the first swallowing efficiency classification or a fourth color different than the third color for the second swallowing efficiency classification.

In an embodiment, the method 500 comprises storing the swallowing safety classification and the swallowing efficiency classification for each of the first plurality of swallowing events in the device 100 in a first profile associated with the first individual (e.g., in the storage element 107). The method 500 can further comprise: screening, with the device 100, a second plurality of swallowing events executed by the first individual subsequent to the first plurality of swallowing events, the first plurality of swallowing events executed on a first beverage having a first viscosity, and the second plurality of swallowing events executed on a second beverage having a second viscosity different than the first viscosity. Preferably, the method 500 comprises storing the swallowing safety classification and the swallowing efficiency classification for each of the first plurality of swallowing events on the device 100 (e.g., the storage element 107) in association with identification of the first beverage in the first profile associated with the first individual; and storing the swallowing safety classification and the swallowing efficiency classification for each of the second plurality of swallowing events on the device 100 (e.g., the storage element 107) in association with identification of the second beverage in the first profile associated with the first individual.

The method 500 can comprise comparing, on the device 100, the swallowing safety and efficiency classifications for the first plurality of swallowing events to the swallowing safety and efficiency classifications for the second plurality of swallowing events. The method 500 can comprise: screening, on the device 100, a second plurality of swallowing events executed by a second individual subsequent to the first plurality of swallowing events; and storing the swallowing safety classification and the swallowing efficiency classification for each of the second plurality of swallowing events in the device 100 in a second profile associated with the second individual (e.g., in the storage element 107).

In an embodiment, the method 500 comprises: screening, on the device 100, a second plurality of swallowing events executed by the first individual subsequent to the first plurality of swallowing events; and comparing, on the device 100, the swallowing safety and efficiency classifications for the first plurality of swallowing events to the swallowing safety and efficiency classifications for the second plurality of swallowing events.

As noted above, the second and particularly preferred embodiment of the screening disclosed herein assigns a probability or percentile to each swallowing event and then uses these probabilities/percentiles to determine a single swallowing safety classification and a single swallowing efficiency classification. The process of assigning a probability or percentile to each swallowing event can be based on comparison of readings from the device 100 to readings from VFSS in order to create an algorithm.

Preferably the readings from the device 100 are compared to readings from VFSS by sequentially administering boluses, measuring simultaneously recorded VFSS readings and accelerometry readings (“Dysphagia Detection System” or “DDS”) for each bolus, identifying the first four matched pairs of each sequence of boluses (i.e., the first four swallowing events measurable by both VFSS and DDS), and then ending administration. The first four matched pairs are used at both patient and bolus levels. Preferably the readings are taken substantially simultaneously between VFSS and DDS. The measurable time difference (e.g., several hundred ms) can be calculated and then taken into account during subsequent calculation to achieve perfect synchronicity between the VFSS and the DDS.

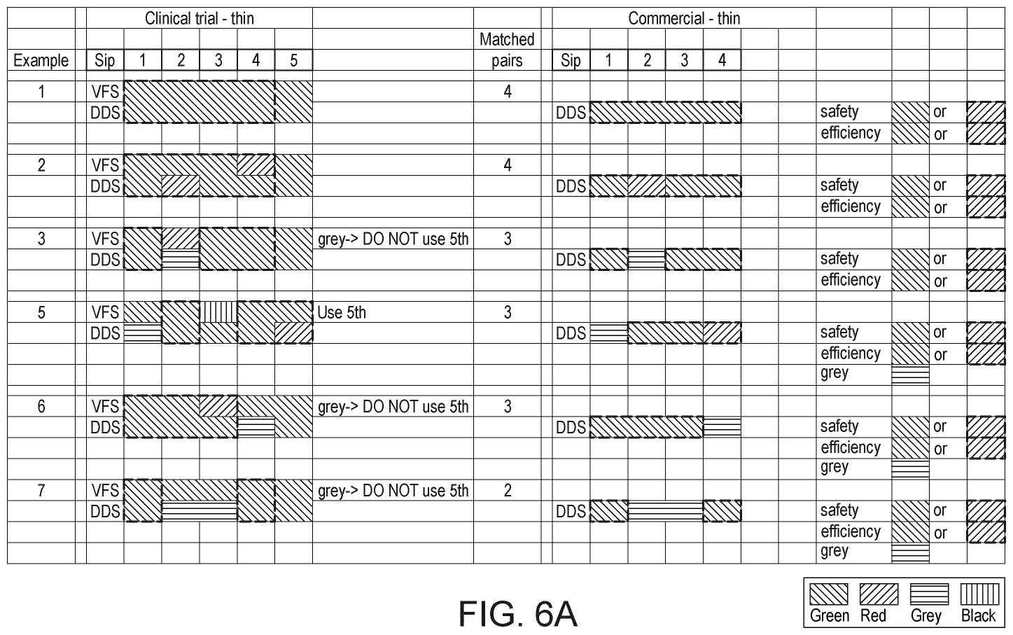

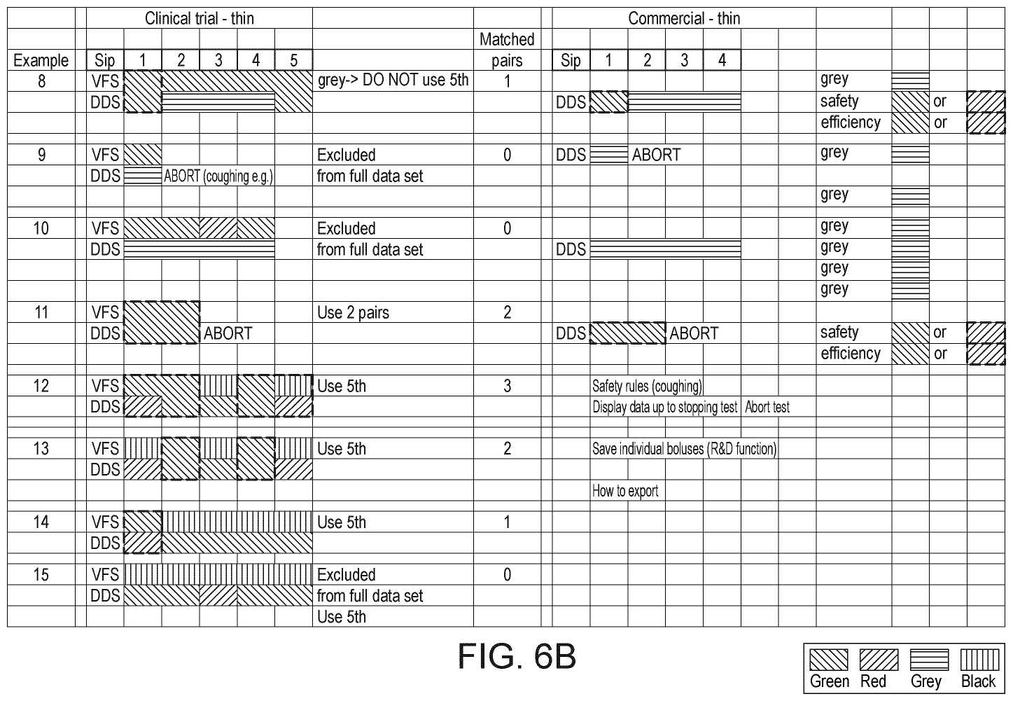

FIGS. 6A and 6B generally illustrate an embodiment of a methodology for identifying the first four matched pairs. The left panel depicts the comparison of the first four matched pairs in a clinical trial in order to determine an algorithm for assigning a probability or percentile to each swallowing event, and the right panel depicts how the algorithm is implemented in a commercial embodiment of the device using the same swallowing outcomes as the clinical study. “Matched pairs” does not mean that the outcome of VFSS and DDS is the same for a swallowing event; rather, “matched pairs” means that the swallowing event is measurable by both VFSS and DDS.

Referring to FIG. 6A, in the case of Example 5 where there is a black event and thus unusable data, the device therefore uses the fifth swallow data if there is a matched pair. If there are more than one black event (e.g., in Example 12 of FIG. 6B), there will only be three matched pairs. In Example 13 of FIG. 6B, there are only two usable matched pairs, etc.

In FIGS. 6A and 6B, the color green identifies a safe and/or efficient swallow, and the color red identifies an unsafe and/or inefficient swallow. The color grey identifies a swallowing event for which DDS could not make a determination, for example an incomplete swallowing event and/or incomplete measurements of a swallowing event. Non-limiting examples include a missing swallow, a signal clipped at start, a signal clipped at end, poor SNR (signal-to-noise ratio), and peak noise. The color black (only used during clinical trial) identifies human error and thus unusable data for the swallowing event.

Preferably the single swallowing safety classification provided by the device 100 is not based on whether more of the swallowing events were classified as safe or more of the swallowing events were classified as unsafe. Similarly, the single swallowing efficiency classification provided by the device 100 are not based on whether more of the swallowing events were classified as efficient or more of the swallowing events were classified as inefficient. To the contrary, more of the swallowing events can be classified as safe, even all classified as safe, but the single swallowing safety classification provided by the device 100 can nevertheless be unsafe swallowing. Similarly, more of the swallowing events can be classified as inefficient, even all classified as safe, but the single swallowing safety classification provided by the device 100 can nevertheless be unsafe swallowing. In this regard, one or more of the swallowing events may be classified as safe and/or efficient nevertheless its accelerometry data may be very close to unsafe and/or inefficient such that the “rolled up” calculation results in the single swallowing safety classification being unsafe and/or the single swallowing efficiency classification being inefficient.

To assign a probability or percentile to each swallowing event, a linear discriminant analysis (LDA) model can be built using the computed features of testing and training accelerometry data. For example, features can be extracted from the accelerometry signal for each swallowing event, such as those related to the head motion and swallow motion components of the signal, the sound direction, the accelerometer velocity and placement estimates, and the entropy of the signal. Each feature can be validated independently of others. The LDA model is a statistical model that is used for classification. The model constructs linear discriminant boundaries in the input feature space to separate a given set of observations into the labelled classes. The model can make the following assumptions: The covariance between feature values across different classes is assumed to be equal and the class conditional distribution is assumed to be multivariate Gaussian in nature. Models can be constructed with different set of features for each combination of bolus type and safety and efficiency problems.

### Preferred Embodiment of the Device

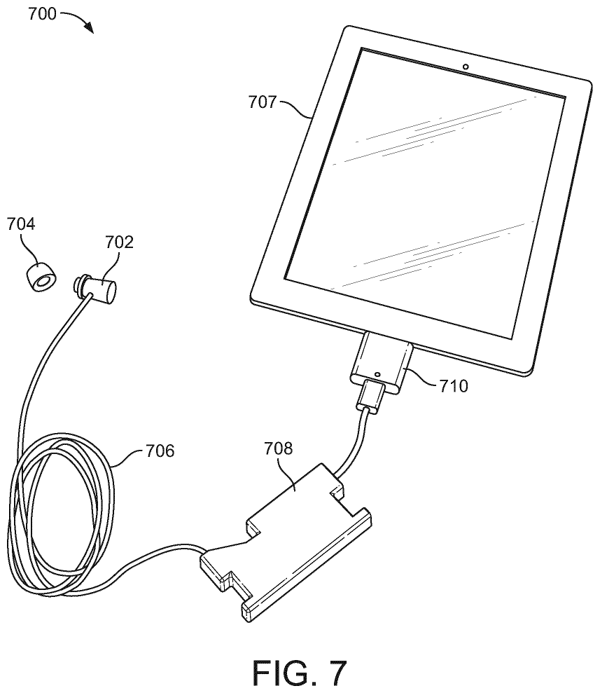

As shown in FIG. 7, a preferred embodiment of the device 100 for screening swallowing safety and swallowing efficiency is a portable, non-invasive device 700 designed for use at the bedside. The portable, non-invasive device 700 can execute either embodiment of the method disclosed above. The portable, non-invasive device 700 can comprise at least three basic components: a sensor unit 702 (“Sensor Head” which can function similarly to the sensor 102); a fixation unit 704 (“Sensor Fixation”) that is preferably single-use and/or disposable; and a mobile tablet and/or handheld unit 707.

The sensor unit 702 preferably comprises a dual-axis accelerometer encased in a housing that can be attached to the front of a patient's neck just below the thyroid cartilage. In an embodiment, the sensor unit 702 comprises a molded plastic housing and a printed circuit board (PCB) sensor unit comprising a 3-axis analog accelerometer.

The fixation unit 704 can adhere the sensor unit 702 to the patient's neck. In an embodiment, the fixation unit 704 comprises an adhesive patch connected to a fixation part such as a molded plastic piece.

The sensor unit 702 can be connected by a first cable 706 to an A/D converter 708 which then can connect by a second cable 710 to the mobile tablet and/or handheld unit 707. The mobile tablet and/or handheld unit 707 can provide one or more of the user interface 104, the input element 105, the processor 106, or the memory element 107, preferably all of these elements.

A dedicated software application on the mobile tablet and/or handheld unit 707 can process the pre-conditioned acceleration data and display the one or more screening results. The portable, non-invasive device 700 can be used for point-in-time assessments or day-to-day by clinicians to assess the changes in a patient's swallowing condition. Any suitable user interface can be appropriate for use with the device 700.

The sensor unit 702 can be attached to the patient's neck to monitor vibrations from the throat. The software application on the mobile tablet and/or handheld unit 707 can compare these vibrations to those typical of healthy patients and patients with impaired swallowing safety. As an additional measure, the application software can be designed to compare the vibrations to those typical of healthy patients and patients with impaired swallowing efficiency. Indications of swallowing safety (risk of penetration-aspiration which describes impaired airway protection) and swallowing efficiency (which describes the ability to clear a bolus through the pharynx in two swallows or less without leaving residue in the throat) can be processed and displayed on the handheld unit.

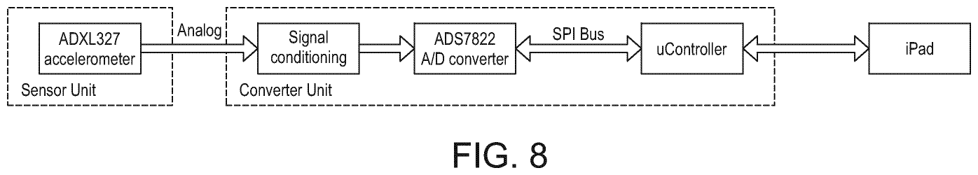

A preferred embodiment of an assessment performed using the device comprises a patient taking a series of sips of a beverage as directed by a clinician who is prompted by the interface software on the tablet. Each sip can be processed and transferred to the application software which can be housed on the mobile tablet. Preferably, the data are analyzed, and the system outputs an assessment of swallowing safety and efficiency. The measurement signal acquisition process is depicted in FIG. 8.

In an embodiment, the A/D converter 708 can be connected via a lead and intended to be placed on a table during the assessment. In another embodiment, the A/D converter 708 assembly can be incorporated in a “necklace” where the A/D converter 708 is suspended on the front of the patient. The A/D converter 708 can be a reusable component.

In another embodiment, a wireless configuration can remove the second cable 710 connecting the mobile tablet and/or handheld unit 707 to the sensor unit 702 and the A/D converter 708. This configuration can allow for wireless transfer of sensor signal data to the mobile tablet and/or handheld unit 707, for example by a Bluetooth wireless connection, and preferably in compliance with all software, data transfer, and electrical safety/EMC standards.

In a preferred embodiment of the tablet, the mobile tablet and/or handheld unit 707 comprises a commercially available tablet that can execute the application software. The incorporation of the mobile tablet and/or handheld unit 707 can be developed in compliance with all mobile applications, software, data transfer, and electrical safety/EMC standards.

In a preferred embodiment, the device 700 (e.g., the mobile tablet and/or handheld unit 707) can comprise an architecture that allows for data transfer and storage, system monitoring, software updating, device management and connection with electronic health record systems.

### Example

The following clinical study presents scientific data developing and supporting one or more embodiments of the device for screening swallowing safety and swallowing efficiency disclosed herein (“Dysphagia Detection System” or “DDS”). Specifically, the tested embodiment of the DDS is a portable, non-invasive device designed for clinical use. A dual-axis accelerometer is contained in the plastic housing of a sensor unit that is attached to the front of a patient's neck just below the palpable lower border of the thyroid cartilage by a single-use, disposable fixation unit. Vibrations are recorded in the superior-inferior and anterior-posterior axes. For this study, the sensor unit was connected via a cable to an A/D converter that then connected to a laptop computer.

The study was a prospective collection and exploration of dual-axis accelerometer signals collected by the DDS during swallows time-synchronized with Videofluroscopic Swallowing Study (VFSS). VFSS was used as the clinical reference standard to determine the true swallowing status of the participating subjects.

The primary objective of the trial was to provide the data required for the development of the classifier algorithm to classify swallow signals captured using a dual-axis accelerometer in terms of impaired swallowing safety and impaired swallowing efficiency in subjects at risk of oropharyngeal dysphagia.

The primary objective consisted of the following sub-objectives:

1. Signal processing and filtering.

2. Segmentation in order to isolate regions of interest. The swallowing events for a particular bolus may further consist of multiple sub-swallows. The objective of the segmentation was to isolate regions in the accelerometer-signal corresponding to each and every sub-swallow.

3. Feature extraction from the accelerometer-signals that would be used as predictors in the classification algorithm.

4. Feature selection and predictive model building.

5. Checking robustness of the final algorithm.

6. Estimating accuracy of the classifier using the area under the curve (AUC) of the receiver operating characteristics (ROC) curve of the classifier using random training-test splits.

7. Providing evidence regarding the equivalence between accelerometer signals for water and thin-barium swallows. This evidence is needed because the trial must be carried out using barium to allow simultaneous assessment by VFSS (the clinical reference method), but the intended use of the DDS is with water.

The secondary objective was to evaluate the impact of sip volume and bolus rheology on accelerometry-based classifiers.

The endpoints related to the primary objective to develop the classifier algorithm:

1. Classification accuracy in terms of area under the curve (AUC) of the receiver operating characteristics (ROC) curve at the bolus level.

2. Optimal threshold for prediction probability at the bolus level and determining corresponding sensitivity and specificity.

Regarding the study procedure, eligible subjects were asked to swallow up to six discrete sips of water during which accelerometry signals were recorded. Immediately following the water sips, subjects underwent VFSS using up to 6 sips of thin barium contrast agent, and up to 3 boluses each of barium thickened to three different consistencies using Resource® Thicken Up Clear (TUC), a xanthan gum containing powder thickener (Nestle Health Science): mildly-thick (1.2 g of TUC/100 ml), moderately-thick (2.4 g of TUC/100 ml), and extremely-thick (3.6 g of TUC/100 ml). Accelerometry signals were simultaneously recorded during the swallows performed in the VFSS.

Water, thin-liquid barium, and mildly-thick barium sips were taken either from a single 7 oz. cup (containing ˜4 oz. of fluid) or as single sips from a series of separate 7 oz. cups (each containing ˜4 oz. of fluid). For water, thin-liquid barium and mildly-thin barium, enrolled subjects were randomized to either a single cup versus series of cups administration method. Intake volumes were measured accordingly. Moderately-thick and extremely-thick barium were taken by spoon. In all cases, sip volume was measured by cup weights after each sip or spoonful.

The videofluoroscopy protocol included stopping criteria intended to maintain patient safety. After the second observation of a particular consistency entering the airway, further boluses of that consistency were terminated. Additionally, after the fifth observation of material entering the airway (regardless of consistency), the videofluoroscopy was stopped.

After 100 subjects had completed the trial, an analysis was conducted to test whether single vs. multiple cups administration methods showed systematic difference in terms of sip volume. Data from 931 boluses from 95 subjects were considered for this analysis. The difference in sip volume between the two methods was found to be statistically non-significant using a linear mixed model, adjusting for pre-test sip volume, gender, and the bolus number. The adjusted difference was estimated as 1.03 ml (SE=1.12, P-value=0.789). The trial continued thereafter using only the single cup method.

Regarding VFSS data collection and analysis, the VFSS exams were performed in the lateral view according to a standard protocol using either continuous fluoroscopy or pulsed fluoroscopy at thirty pulses per second, and were recorded on a laptop computer at a frame rate of thirty frames per second. Videofluoroscopy recordings were transferred electronically to the Swallowing Rehabilitation Research Laboratory (Toronto Rehabilitation Institute—University Health Network) for processing and analysis. The analysis process involved an initial image quality review and the identification of time codes corresponding to the beginning and end of events for each bolus. The recordings were then spliced into bolus-level clips in Matlab. Each bolus clip was assigned a random number for rating purposes. A total of 4229 bolus clips were analyzed in duplicate by pairs of raters. Raters were licensed speech-language pathologists (SLPs) who had been trained to be competent in each rating task according to a standard operating procedure.

Swallowing safety was categorized for all subswallows for each bolus using the 8-point Penetration-Aspiration Scale (PAS). Scores of 1 and 2 on this scale reflect no safety concern. Scores of 3-5 are classified as “penetration,” whereby material enters the supraglottic space but does not travel below the true vocal folds. Scores of 6-8 are classified as “aspiration”, whereby material travels below the true vocal folds. A score of 8 indicates “silent aspiration” where there is no cough or throat clearing response from the patient. The PAS scores were subsequently reduced to a binary swallowing safety score, with scores of 1 and 2 classified as “safe” and scores of 3 and higher classified as “unsafe.”

Swallowing efficiency describes the degree to which a person is able to swallow and clear a bolus through the pharynx within two swallows or less without leaving residue. In this study, three criteria were used to label the swallowing of a bolus as inefficient:

a) Any case where more than two subswallows were seen for a bolus was classified as inefficient.

b) The accumulation of vallecular residue above a subjective rating of 1 (corresponding to residue subjectively judged to be filling more than 50% of the available space in the valleculae) was considered inefficient.

c) The accumulation of pyriform sinus residue above subjective rating of 1 (corresponding to residue subjectively judged to be filling more than 50% of the available space in the pyriform sinuses) was considered inefficient.

In addition to ratings of residue (0=none, 1=up to 50% full, 2=>50% full), pixel-based measurements of residue were made, providing a more precise calculation of the degree to which each space was full (%-full) and allowing calculation of residue severity according to the Normalized Residue Ratio Scale, which incorporates normalization of residue and spatial housing measures to an anatomical scalar reference (the length of the C2-C4 cervical spine).

VFSS classification was carried out at the bolus level, i.e., each bolus was rated independently by VFSS raters at the central lab. The raters were blinded to the subject identification. The results at bolus level were then rolled-up to patient level. Any single event of airway invasion across the boluses for a particular consistency would result in a participant level result of “unsafe” for that consistency. Similarly, any single event of swallowing inefficiency across the boluses for a given consistency would result in a participant level result of “inefficient” for that consistency.

The development of the classification algorithm can be described in terms of the following components: 1. Pre-processing of the DDS signals; 2. Segmentation to isolate the region of swallow activity; 3. Feature extraction; and 4. Running classification experiments to estimate predictive accuracy of the classifier.

1. Signal Pre-Processing

The accelerometry signals were collected at a sampling frequency of 10 kHz. It has been shown that the majority of signal power in dual-axis swallowing accelerometry observations is concentrated below 100 Hz. In this study, the accelerometry signals were denoised via 10-level wavelet decomposition with Daubechies-8 mother wavelets, and reconstructed with soft-thresholding.

The approximation and detail wavelet coefficients were also used to extract the signal components corresponding to head motion and to identify vocalization segments within the captured swallowing signals.

Specifically, the approximation wavelet coefficients at level 10 were used to reconstruct the signal components containing frequencies less than 5 Hz that are reported to be the frequency content characterizing head motion. To isolate the signal component with frequency content characterizing vocalization, everything except the detail wavelet coefficients corresponding to the frequency range 40-650 Hz (detail coefficients of level 5 to 8) was suppressed.

Within the extracted vocalization component of the signal (40-650 Hz content), the active segments were identified via a peak search, and those segments with duration between 0.4 to 1 seconds were identified as vocalization segments.

2. Segmentation

Each bolus consumed by a participant may have been ingested via one or more subswallows. Segmentation involves identifying for each bolus the location of one or multiple swallow activity regions. Segmentation was carried out independently by each of the three teams and later merged using the union of the segments recognized by the three segmentation algorithms.

The unsupervised algorithm divided the bolus signal into smaller segments and removed any segments with very large signal spikes, eliminating artifacts from signal sources unrelated to swallowing. Candidate swallows were identified from the remaining segments via a fuzzy c-means clustering method that separated segments with high standard deviation from those with low standard deviation. Signals from the S-I and A-P axes (the two orthogonal axes of the sensor) were segmented separately, and the intersection of the candidate swallow segments from each axis comprised the final segmentation outputs.

The supervised algorithm involved dividing bolus signals into segments of equal length of 1.3 second long segments with the possibility of an overlap between consecutive segments of 0.3 seconds and then using template-matching. A random split of the data resulted in small training and test sets. Segments were extracted from between the VFSS start and stop times and also from the non-swallow regions. These segments were called swallow templates and non-swallow templates, respectively. For test boluses, the swallow segments were then identified using a measure of similarity with swallow/non-swallow templates. Finally, adaptive thresholding and segment merging (for very short neighboring segments) was done before finalizing the swallow segment for each bolus.

Another unsupervised approach involved polar transformation of the S-I and A-P axis signals, a low-pass filter of Hz and a high-pass filter of 0.5 Hz, using hidden Markov models (HMM) to find initial swallow-segments and using the EM-algorithm to classify different segments into swallow or non-swallow regions based on correlation between the two axes and signal amplitude.

3. Feature Extraction and Selection

Feature extraction algorithms were implemented in Matlab by two independent signal analysis groups. A total of 100+ different features were calculated for each bolus signal following segmentation. Features with the ability to discriminate between healthy and impaired swallow were selected based on experience. The features include some that are based on S-I and A-P axes separately, as well as those that combined information from both axes. Time, frequency and time-frequency domain features were included for consideration.

The 100+ different features were reduced to a minimal set of salient features using a regularized binomial regression with elastic-net penalty and the ordering of importance was obtained.

Following this reduction, Linear Discriminant Analysis (LDA) models and Support Vector Machine (SVM) models were fitted to the training sets and used for prediction on the test sets. This process was carried out 1,000 times. For each training-test split, the AUC of the ROC curves for increasing number of features was traced for the training as well as the test set in order to observe over-fitting tendencies. The number of features to be included for each classification model was determined in a conservative fashion to avoid over-fitting.

4. Estimating Classification Accuracy

Following feature selection, classification accuracy of the LDA and other models were calculated using cross-validation that was repeated 10,000 times. During each run of the cross-validation, bolus signals from 20% of the participants were set aside to be the testing set, and the boluses from the remaining 80% of participants were used to train the classifier. This random splitting was stratified by the patient status derived from the VFS results. The classifier was trained using Linear Discriminant Analysis methodology. Using the same set of features, other classifier models such as SVM were also tested for accuracy.

Threshold tuning was applied to the resulting classifier to identify the probability threshold that would yield bolus-level accuracies in the training set that were most in line with the target sensitivity/specificity levels of 90%/60%. The testing set was subsequently passed through the classifier with the tuned threshold level applied, and the resulting bolus-level sensitivity and specificity were calculated. The bolus-level accuracies of the testing sets across all 10,000 runs were averaged to provide the final sensitivity and specificity levels for the classifier.

Results

Patient Characteristics

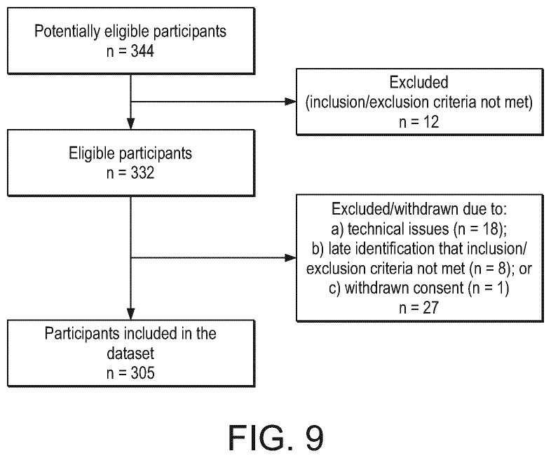

344 subjects were assessed for eligibility to participate, and 12 did not meet inclusion/exclusion criteria. 305 of the initially enrolled 344 subjects had at least 2 boluses with complete data (i.e. simultaneously recorded VFSS and DDS signals). FIG. 9 reports the flow of participants through the study.

Demographic characteristics are outlined in Table 1 below. The mean age of study participants was 70 years, and 50% were women. A total of 107 (32.2%) patients had stroke, and 18 (5.4%) had acute brain injury other than stroke. 207 (62.3%) of participants are grouped under “Others” representing all enrolled patients above 50 years who presented with different medical conditions other than acute brain injury/stroke.

TABLE 1
Demographic characteristics of the subjects who underwent
videofluoroscopy by diagnostic subgroup. The data are shown
according to the following convention x ± s represents the
mean ± one standard deviation.

| | | | Other | | |
|---|---|---|---|---|---|
| | | | Brain | Other, | |
| | | Stroke | Injury | Aged ≥50 | Combined |
| | N | (N = 107) | (N = 18) | (N = 207) | (N = 332) |
| Sex | 332 | | | | |
| Women | | 48 (45%) | 4 (22%) | 113 (55%) | 165 (50%) |
| Men | | 59 (55%) | 14 (78%) | 94 (45%) | 167 (50%) |
| Age | 332 | 70 ± 14 | 67 ± 11 | 73 ± 11 | 72 ± 12 |
| VFSS | 332 | | | | |
| recorded | | | | | |

In total, after exclusions were removed, VFSS results were available for 305 participants. The videofluoroscopy protocol included stopping criteria intended to maintain patient safety, therefore not all participants completed the full protocol of six thin liquid boluses, three mildly-thick boluses, three moderately thick boluses and three extremely-thick boluses. Additionally, video quality issues (such as shoulder shadows occluding the view, and issues with turning the fluoroscopy on late or off early) meant that some clips could not be rated.

Swallowing Safety and Efficiency

1,730 thin swallows (bolus), 872 mildly thick boluses, 833 moderately thick boluses and 794 extremely thick boluses were analyzed for swallowing safety and efficiency. The prevalence of impaired swallowing safety or efficiency at participant level was determined from VFSS bolus analysis as follows:
Participants were considered to have impaired swallowing or efficiency on thin stimuli if at least one impaired bolus in the series of one to six swallows was impaired for safety or efficiency respectively.
Participants were considered to have impaired swallowing or efficiency on mild, moderate or extremely thick stimuli if a least one impaired bolus in the series of one to three swallows for the respective consistency was impaired for safety or efficiency respectively.

Table 2 below shows the prevalence of unsafe swallows and efficiency issues at bolus level and at participant level, by stimulus type. The data demonstrated that swallowing safety issues decreased substantially with increased viscosity of stimuli, resulting in less available data for classifier development for moderate and extremely thick consistencies.

TABLE 2
Prevalence of impaired swallowing safety and impaired swallowing efficiency at the
bolus and subject level, by stimulus type.

| | | | Impaired Safety | | | | Impaired Efficiency | | | |
|---|---|---|---|---|---|---|---|---|---|---|
| | Number of Data- | | Bolus | | Participant | | Bolus | | Participant | |
| Stimulus | points Available | | Level | | Level | | Level | | Level | |
| Consistency | Boluses | Participants | n | % | n | % | n | % | n | % |
| Thin (1-6 | 1730 | 305 | 125 | 7.2% | 70 | 23.0% | 115 | 6.7% | 60 | 19.7% |
| boluses) | | | | | | | | | | |
| Mildly-thick | 872 | 302 | 51 | 5.9% | 42 | 13.9% | 86 | 9.9% | 54 | 17.9% |
| Moderately- | 833 | 281 | 17 | 2.0% | 14 | 5.0% | 75 | 8.9% | 51 | 18.2% |
| thick | | | | | | | | | | |
| Extremely-thick | 794 | 268 | 11 | 1.4% | 10 | 3.7% | 67 | 8.4% | 48 | 17.9% |

Swallow efficiency is not usually addressed by dysphagia screening which is focused on safety and risk of aspiration. Individuals who have impaired swallowing efficiency may require longer to complete a meal and are thought to be at risk for malnutrition. The data suggests that swallowing inefficiency is a prevalent and important issue which requires further study.

The difference between the prevalence of impaired safety at bolus vs. participant level as determined by VFSS is explained by intra-patient variability of swallowing: in patients with dysphagia not all swallows are impaired. Such intra-patient variability in swallowing (between boluses in the series of swallows) is very important to consider for the design of the DDS validation trial: focusing on bolus level accuracy and using simultaneous recording of DDS and VFSS are both important to ensure reliable validation.

Optimal Number of Boluses to Detect Swallowing Safety

There is no consensus among experts on what is the optimal number of boluses for swallow assessment.

In order to determine the optimal number of boluses to detect swallowing safety for thin stimulus at patient level, the cumulative percentage of detected impairment of swallowing safety at the participants level was analyzed (Table 3 below). The cumulative percentage after four boluses was 22.16% going up to 25.6% after six boluses with a flattening curve of gain in cumulative percentage.

TABLE 3
Participant-level percentage of detected impairment of swallowing
safety on thin stimuli.

| | N at risk of | | No Participants | Cumulative percentage |
|---|---|---|---|---|
| | first | | with | of detected |
| Bolus | detection | N · event | impairment (%) | impairment (%) |
| 1 | 305 | 23 | 7.54 | 7.54 |
| 2 | 282 | 21 | 7.45 | 14.99 |
| 3 | 257 | 9 | 3.50 | 18.49 |
| 4 | 245 | 9 | 3.67 | 22.16 |
| 5 | 235 | 3 | 1.28 | 23.44 |
| 6 | 230 | 5 | 2.17 | 25.61 |

Given this finding, the mean predicted probability of impairment was summarized at the subject level using up to 4 boluses for thin and up to 3 boluses for other consistencies. The receiver operator curve at subject level was obtained by comparing these mean predicted probabilities with the subject level class label obtained using the “at least one positive” roll-up rule on the VFSS binary data. Thus, if the VFSS showed a problem on at least one bolus of a given consistency, then that patient was considered to have impaired swallowing function on that consistency. As will be noted below, the prevalence of impaired swallowing safety decreased substantially with the thicker stimuli. Given the limited availability of impaired swallowing safety data with these consistencies, it was decided to develop one classifier for the combined moderately and extremely thick stimuli (henceforth referred to as moderately/extremely thick).

Taking into consideration incremental gain in detecting impaired swallowing vs. radiation exposure during VFSS and the objective to develop a device as an easy-to-use, practical bedside tool to detect impaired swallow, four were used boluses to build the classifier. DDS will allow for up to 4 boluses testing for thin stimulus.

In various embodiments, the classifier algorithm disclosed herein can be built using an optimal number of four boluses for improved screening of any suitable dysphagia device. As discussed above, a multitude of different ways monitor and detect oropharyngeal dysphagia, including but not limited to the preferred method of using the DDS employed in the specific exploratory study and illustrated in FIG. 8. Once built, the classifier can be used to accurately and safely assess whether a patient has impaired swallowing, regardless of whether the dysphagia device is using VFSS, FEES, sonar, or any other technique.

For other stimuli (mild, moderate and extremely thick), the classifier was built using the three-boluses approach available as per study protocol. Taking into consideration the learnings for thin stimulus, the plan for the validation trial includes data collection for four boluses for each stimulus.

DDS Classification Accuracy

Results from 10,000 random splits of the data into training and test sets, stratified by the patient's disease status as determined using the VFS class labels, are given hereafter. The main classifier is based on the LDA model. Other classifiers including SVMs have also been fitted and tested for accuracy in order to check robustness. Here results are presented for the LDA model by consistency. Sensitivity and specificity are computed for each random training-test split and are based on optimized thresholds, i.e., the point on the ROC curve that is the minimum distance from the targeted point (sensitivity=0.9, 1−specificity=0.4) corresponding to 90% sensitivity and 60% specificity. The AUC of the ROC curve at the bolus level is the primary endpoint which is independent of any thresholds. Tables 4a and 4b below summarize the estimated accuracy of the LDA classifier using 10,000 random training-test splits of the data (Table 4a is swallowing safety, Table 4b is swallowing efficiency). For the three thick consistencies, the number of features and the particular features used are different. However, there are overlapping features, and the non-overlapping features are strongly correlated with at least one feature used in the other classifier.

TABLE 4a
Classifier Accuracy for detecting Swallow Safety Problems by
Consistency at Patient Level

| | Bolus level | Participant | | |
|---|---|---|---|---|
| | AUC (%) | level AUC | Sensitivity | Specificity |
| | Mean ± | (%) Mean ± | (%) | (%) |
| Consistency | SD | SD | Mean ± SD | Mean ± SD |
| Thin | 80.9 ± 5.9 | 81.5 ± 6.1 | 90.4 ± 7.7 | 60.0 ± 7.8 |
| Mildly Thick | 83.9 ± 5.6 | 83.6 ± 5.9 | 92.7 ± 8.7 | 59.9 ± 7.5 |
| Moderately Thick* | 78.9 ± 11.9 | 79.7 ± 15.1 | 89.1 ± 22.0 | 59.6 ± 7.6 |

TABLE 4b
Classifier Accuracy for detecting Swallow Efficiency Problems by
Consistency at Patient Level

| | | Participant | | |
|---|---|---|---|---|
| | Bolus level | level AUC | Sensitivity | Specificity |
| | AUC (%) | (%) | (%) | (%) |
| Consistency | Mean ± SD | Mean ± SD | Mean ± SD | Mean ± SD |
| Thin | 76.7 ± 6.4 | 77.7 ± 7.5 | 82.3 ± 11.0 | 59.2 ± 7.9 |
| Mildly Thick | 80.1 ± 6.0 | 78.0 ± 7.7 | 82.4 ± 11.0 | 59.6 ± 7.9 |
| Moderately Thick | 73.3 ± 7.1 | 71.9 ± 8.3 | 79.3 ± 11.7 | 59.3 ± 8.2 |

Signals with high level of noise: All accelerometry signals collected were classed as “safe” or “unsafe” as shown in Tables 4a and 4b. However, some of these signals exhibited a high level of noise, making it difficult to implement the segmentation.

For the validation trial, therefore, the idea of “gray” signals is introduced, defined by the level of spectral entropy (disorder in the signal). This idea was expected to result in at most 5% of the signals being classified as “gray,” where the patient should be referred for further evaluation.

Thresholding and variability on sensitivity & specificity estimates: Two factors contribute to the rather high variability in sensitivity and specificity estimates:

1. Small test sets: 70 out of 305 participants had shown swallowing safety problems in at least one bolus with thin consistency boluses. The random training-test 80-20 splits resulted in a small number of subjects with problems.

2. Optimized thresholding minimizing the distance between each ROC curve and the targeted performance points (0.4, 0.9) was carried out separately for each random split. Again due to small test sets there was a large variability in the thresholds which further contributed to the variability seen in the sensitivity and specificity estimates.

Water-Thin Equivalence: The main objective of this trial was to use the simultaneously-collected accelerometer signals and VFSS on thin-barium swallows to develop a classifier algorithm that could predict swallow impairments (safety and/or efficiency) based on the accelerometer signals alone. However, the main intended use of the device in the future is to detect swallowing impairment using water swallows. For this reason, accelerometer signals were collected for water swallows but without the simultaneous VFSS.

An equivalence test based on data from the first eighty participants who had at least two swallows available for both water and thin-barium was carried out. An equivalence-margin of 10% was used as the limit of agreement as commonly done. A segmentation algorithm based on Hidden-Markov-Models was used to extract the main swallow profile for each swallow for each subject. In order to reduce the dual axis problem to the univariate case, the function (x, y) [symbol] x2+y2 was used. Following peak alignment, a fixed time-width of one-second centered at the peak was then extracted. A functional mixed additive model proposed by Scheipl, Staicu and Greven was then fitted to the data with random effects smooth terms for subject. 95% point-wise confidence intervals were computed for the difference between mean swallow profiles for water and thin-barium. Results showed that the 95% point-wise confidence intervals for the difference were within ±10%.

### Conclusions and Implications for Validation Trial Design

Swallowing Safety using thin stimulus:

a. An AUC of 0.82 of the ROC curve at bolus level was obtained for detecting swallow safety problems

b. Data on cumulative percentage of detected impairment of swallowing safety suggest that four boluses is an optimal number to detect a problem of swallowing safety

c. Sensitivity and specificity close to targeted values of 90% and 60% (86.7% and 60.4% respectively) were reached for thin stimuli

d. Since validation with VFSS was not possible with water, a test of equivalence was performed. The test showed that the mean swallow accelerometry profiles for water and thin-barium can be considered equivalent for a given participant.

Swallowing Safety using thicker stimuli:

a. An AUC of 0.83 of the ROC curve was obtained for detecting swallow safety problems for mild stimulus. The AUC for the moderate & extremely thick consistency reached 0.76 and 0.87 despite a limited amount of available data.

b. Sensitivity and specificity of 87.5% and 60.4% were obtained for mild stimulus, and 3.3% and 61.1% for moderate. Results for extremely thick stimulus were unreliable due to very low prevalence.

The results revealed that participants with swallowing impairment do not necessarily exhibit impaired swallow in every bolus in a series of swallows. Therefore the simultaneous measures by DDS and the clinical reference method (VFSS), as performed in the trial, are critical for the future validation trial.

The data indicates that four boluses are needed to detect impaired swallowing safety on thin stimuli and suggests that any method using fewer than four boluses could miss the impairment. This holds both for non-radiographic and videofluoroscopy protocols. The data also suggests that collecting more than four thin boluses adds marginal incremental benefit for detecting impaired safety. This is useful information because the collection of more than four boluses in videofluoroscopy increases radiation exposure and may induce fatigue.

The majority of swallow screening protocols in current use focus on signs of impaired safety and risk of aspiration, without consideration of swallow efficiency. The data in the study shows that swallowing inefficiency is frequent and important. Furthermore, the data suggests that residue can be common on thin liquids.

No serious adverse events related to the device or the swallowing protocol were observed.

The convention used in this project was to classify swallowing safety and efficiency based on the worst score assigned across the subswallows for a particular bolus. However, safety and efficiency parameters can be rolled-up to provide an overall score for a given bolus. For the primary outcome of detecting impaired swallowing safety on thin liquids, a mean AUC of 80.9% on the ROC was obtained at the bolus level. When rolled up to the subject level, the mean AUC was 81.5%, with sensitivity (i.e. true positive rate) of 90.4% and specificity (i.e., true negative rate) of 60.0%, respectively. Classifier performance was similarly strong for detecting impaired swallowing safety on thicker consistencies. The efficiency classifiers achieved sensitivities of ˜80% and specificities of 60% across the consistencies tested.

The accelerometry signal classifier algorithms developed in this study allow the detection of impaired swallowing with high accuracy. Importantly, this provides an automated, non-invasive means of swallowing evaluation at the bedside.

Various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. An integrated device for screening swallowing safety and swallowing efficiency, the device comprising:
a processor configured to (i) receive first vibrational data for a first plurality of swallowing events executed successively by a first individual, (ii) compare swallowing data selected from the group consisting of at least a portion of the first vibrational data, at least a portion of second vibrational data derived from the first vibrational data, and a combination thereof against preset classification criteria defined for each of swallowing safety and swallowing efficiency, (iii) assign a swallowing safety probability and a swallowing efficiency probability to each of the first plurality of swallowing events, each of the first plurality of swallowing events is assigned the corresponding swallowing safety probability and the corresponding swallowing efficiency probability independently from the other swallowing events to provide independent point measurements for the first plurality of swallowing events, (iv) determine a swallowing safety classification for the first plurality of swallowing events based at least partially on the swallowing safety probability of each of the first plurality of swallowing events, the swallowing safety classification is identified from at least one predetermined swallowing safety classification, and (v) determine a swallowing efficiency classification for the first plurality of swallowing events based at least partially on the swallowing efficiency probability of each of the first plurality of swallowing events, the swallowing efficiency classification is identified from at least one predetermined swallowing efficiency classification; and
a user interface configured to provide one or more first outputs comprising at least one of audio and/or graphics that identify the swallowing safety classification and the swallowing efficiency classification for the first plurality of swallowing events.

2. The integrated screening device of claim 1 wherein the swallowing safety probability and the swallowing efficiency probability are assigned to the corresponding swallowing event in real-time relative to the corresponding swallowing event.

3. The integrated screening device of claim 1 wherein the one or more first outputs by the user interface each are real-time relative to completion of the first plurality of swallowing events.

4. The integrated screening device of claim 1 further comprising an accelerometer communicatively connected to the processor to provide the first vibrational data.

5. The integrated screening device of claim 1 further comprising a housing, and the processor and the user interface each have a position individually selected from the group consisting of: within the housing, mechanically connected to the housing, and a combination thereof.

6. The integrated screening device of claim 1 wherein the swallowing safety classification is the single swallowing safety classification for the first plurality of swallowing events, and the swallowing efficiency classification is the single swallowing efficiency classification for the first plurality of swallowing events.

7. The integrated screening device of claim 1 wherein the processor is configured to use the user interface to provide one or more second user outputs comprising at least one of audio and/or graphics that instruct administration of a plurality of doses of beverage, and each of the first plurality of swallowing events correspond to one of the plurality of doses of beverage.

8-13. (canceled)

14. The integrated screening device of claim 1 comprising a memory element configured to store the swallowing safety classification and the swallowing efficiency classification for the first plurality of swallowing events in a first profile associated with the first individual.

15-17. (canceled)

18. The integrated screening device of claim 1 wherein the processor is configured to screen a second plurality of swallowing events executed by the first individual at least one day after the first plurality of swallowing events, and the processor is configured to compare the swallowing safety classification for the first plurality of swallowing events to a swallowing safety classification for the second plurality of swallowing events and compare the swallowing efficiency classification for the first plurality of swallowing events to a swallowing efficiency classification for the second plurality of swallowing events.

19. (canceled)

20. A method of screening swallowing safety and swallowing efficiency, the method comprising:
receiving, on a device comprising a processor, first vibrational data for a first plurality of swallowing events executed successively by a first individual;
comparing, on the device, swallowing data selected from the group consisting of at least a portion of the first vibrational data, at least a portion of second vibrational data derived from the first vibrational data, and a combination thereof against preset classification criteria defined for each of swallowing safety and swallowing efficiency;
determining a swallowing safety probability and a swallowing efficiency probability for each of the first plurality of swallowing events based at least partially on the comparing of the swallowing data against the preset classification criteria, each of the first plurality of swallowing events is assigned the corresponding swallowing safety probability and the corresponding swallowing efficiency probability independently from the other swallowing events to provide independent point measurements for the first plurality of swallowing events;
determining a swallowing safety classification for the first plurality of swallowing events based at least partially on the swallowing safety probability of each of the first plurality of swallowing events, the swallowing safety classification is identified from at least one predetermined swallowing safety classification;
determining a swallowing efficiency classification for the first plurality of swallowing events based at least partially on the swallowing efficiency probability of each of the first plurality of swallowing events, the swallowing efficiency classification is identified from at least one predetermined swallowing efficiency classification; and
producing, from the device, one or more first outputs comprising at least one of audio and/or graphics that identify the swallowing safety classification and the swallowing efficiency classification for the first plurality of swallowing events.

21. The method of claim 20 wherein the determining by the device of the swallowing safety probability and a swallowing efficiency probability is real-time relative to the corresponding swallowing event.

22. The method of claim 20 wherein the one or more first outputs identifying of the swallowing safety classification and the swallowing efficiency classification for the first plurality of swallowing events is real-time relative to completion of the first plurality of swallowing events.

23. The method of claim 20 comprising transmitting the first vibrational data to the device from an accelerometer communicatively connected to the device.

24. The method of claim 20 wherein the device comprises a housing and comprises a user interface that provides the one or more first outputs, and the processor and the user interface each have a position individually selected from the group consisting of: within the housing, mechanically connected to the housing, and a combination thereof.

25. The method of claim 20 wherein the swallowing safety classification is the single swallowing safety classification for the first plurality of swallowing events, and the swallowing efficiency classification is the single swallowing efficiency classification for the first plurality of swallowing events.

26. The method of claim 20 comprising producing, from the device, one or more second outputs comprising at least one of audio and/or graphics that instruct administration of a plurality of doses of beverage, and the first plurality of swallowing events each correspond to one of the plurality of doses of beverage.

27-30. (canceled)

31. The method of claim 20 wherein:
the at least one predetermined swallowing safety classification comprises a first swallowing safety classification indicative of a safe event and a second swallowing safety classification indicative of an unsafe event,
the at least one predetermined swallowing efficiency classification comprises a first swallowing efficiency classification indicative of an efficient event and a second swallowing efficiency classification indicative of an inefficient event, and
the one or more first outputs comprise at least one icon for each of the first plurality of swallowing events, the at least one icon is displayed on a user interface of the device, at least a portion of the at least one icon is a first color for the first swallowing safety classification or a second color different than the first color for the second swallowing safety classification, and at least a portion of the at least one icon is a third color for the first swallowing efficiency classification or a fourth color different than the third color for the second swallowing efficiency classification.

32-37. (canceled)

38. The method of claim 20 wherein the first individual is continuously monitored over a time period such that the device is not removed from the first individual during the time period, the first plurality of swallowing events is executed on a first beverage having a first viscosity and consumed by the first individual during a first portion of the time period, and a second plurality of swallowing events is executed on a second beverage having a second viscosity different than the first viscosity and consumed by the first individual during a second portion of the time period that is before or after the first time period.

39. (canceled)
